(19) United States
(12) Patent Application Publication
(10) Pub. No.: US 2021/0277121 A1
(43) Pub. Date: Sep. 9, 2021

(54) DUAL TARGETING ANTIGEN BINDING MOLECULE

(71) Applicant: MAB-LEGEND BIOTECH CO., LTD., Shanghai (CN)

(72) Inventors: Shaoxiong WANG, Shanghai (CN); Jing ZHAO, Shanghai (CN); Liming HUANG, Shanghai (CN); Ming WANG, Shanghai (CN); Juehua XU, Shanghai (CN); John L. XU, Shanghai (CN); Shanshan ZOU, Shanghai (CN)

(21) Appl. No.: 17/280,726

(22) PCT Filed: Sep. 29, 2018
(86) PCT No.: PCT/CN2018/108700

§ 371 Date: Mar. 26, 2021

(51) Int. Cl. C07K 16/28

(52) U.S. Cl. CPC C07K 16/2809; C07K 16/2887; C07K 2317/55; C07K 2317/622; A61K 2039/505; C07K 2317/71; C07K 2317/31; C07K 2317/64; C07K 2317/526

(57) ABSTRACT

The present invention is related to a dual targeting antigen binding molecule, a pharmaceutical composition comprising the dual targeting antigen binding molecule and the uses thereof for the treatment of diseases. Additionally, the present invention is also involved in a method for producing the dual targeting antigen binding molecule.

### CROSS-REFERENCE TO RELATED APPLICATIONS

This application claims priority to PCT application No. PCT/CN2018/108700, filed on Sep. 29, 2018, hereby incorporated herein by reference in its entirety.

### FIELD OF THE INVENTION

The present invention is related to a dual targeting antigen binding molecule, a pharmaceutical composition comprising the dual targeting antigen binding molecule and the uses thereof for the treatment of diseases. Additionally, the present invention is also involved in a method for producing the dual targeting antigen binding molecule.

### BACKGROUND OF THE INVENTION

Dual targeting antigen binding molecules target two different kinds of antigens, for example, the CD3 molecule of T cell and a tumor specific antigen of cancer cell, hold great promises for cancer therapy. Among these molecules, there are bispecific antibodies which had been launched into the market. However, up to now, the treatment effect of these bispecific antibodies is not as satisfactory as expected. Taking the Catumaxomab (EpCAM×CD3 bispecific antibody) as an example, which had been withdrawn from the market in view of significant side effects resulting from off-target ADCC effect. Another example, blinatumomab (CD19×CD3 bispecific T cell engager), due to its short half-life and inconvenient dosing regimen, the enthusiasm of applying this medicament to patients has been frustrated a lot. The present invention is aiming to develop a new generation of dual targeting antigen binding molecules that overcome these issues.

### SUMMARY OF THE INVENTION

The present invention is related to a new generation of dual targeting antigen binding molecules which, on one hand, exhibit good effects of binding and killing target cells, and on another hand, display regular IgG pharmacokinetics (e.g., long plasma half-life) with reduced Fc-mediated side effects. Such molecules as provided by the present invention would to some extent satisfy the clinical needs on dual targeting antigen binding molecules.

Specifically, in one aspect, as provided by the present invention is a dual targeting antigen binding molecule, comprising a first antigen binding moiety capable of specific binding to a T cell-activating antigen, and a second antigen binding moiety capable of specific binding to a target cell antigen, wherein the first antigen binding moiety comprises a scFv and the second antigen binding moiety comprises a first Fab and a second Fab. In certain embodiments, the first Fab and the second Fab bind the same target cell antigen. In certain embodiments, the first Fab and the second Fab bind different epitopes of the same target cell antigen. In certain embodiments, the first Fab and the second Fab bind the same epitope of a target cell antigen. In certain embodiments, the first Fab and the second Fab are derived from the same antibody. In certain embodiments, the first Fab and the second Fab are derived from different antibodies binding to the same target cell antigen.

In certain embodiments, in the dual targeting antigen binding molecule of the present invention, the scFv comprises a variable region of heavy chain (V_{H}) and a variable region of light chain (V_{L}) from the N-terminus to C-terminus of the scFv, or a variable region of light chain (V_{L}) and a variable region of heavy chain (V_{H}) from the N-terminus to C-terminus of the scFv. In preferable embodiments, the dual targeting antigen binding molecule further comprises an Fc domain consisting of a first and a second subunit capable of stable association. In certain embodiments, the second antigen binding moiety comprises a first Fab fused at the C-terminus of its Fab heavy chain to the N-terminus of the variable region of light chain (V_{L}) of the scFv or the N-terminus of the variable region of heavy chain (V_{H}) of the scFv, and the C-terminus of the scFv is connected to one of the first and second subunit of the Fc domain, and the second antigen binding moiety comprises a second Fab fused at the C-terminus of its Fab heavy chain to the other subunit of the Fc domain. In particular embodiments, the first Fab fused at the C-terminus of the Fab heavy chain to the N-terminus of the variable region of heavy chain (V_{H}) of the scFv, or the first Fab fused at the C-terminus of the Fab heavy chain to the N-terminus of the variable region of light chain (V_{L}) of the scFv.

In certain embodiments, in the dual targeting antigen binding molecule of the present invention, the second antigen binding moiety comprises a first Fab fused at the N-terminus of its Fab heavy chain to the C-terminus of the variable region of light chain (V_{L}) of the scFv or the C-terminus of the variable region of heavy chain (V_{H}) of the scFv, and the C-terminus of the Fab heavy chain of the first Fab is connected to one of the first and second subunit of the Fc domain, and the second antigen binding moiety comprises a second Fab fused at the C-terminus of its Fab heavy chain to the other subunit of the Fc domain. In particular embodiments, the first Fab fused at its N-terminus of the Fab heavy chain to the C-terminus of the variable region of heavy chain (V_{H}) of the scFv or wherein the first Fab fused at its N-terminus of the Fab heavy chain to the C-terminus of the variable region of light chain (V_{L}) of the scFv.

In certain embodiments, the dual targeting antigen binding molecule of the present invention comprises only one antigen binding moiety capable of specific binding to a T cell-activating antigen.

According to any of the above embodiments, components of the dual targeting antigen binding molecule of the present invention, for example, the first antigen binding moiety, the second antigen binding moiety, the variable region of light chain (V_{L}) of the scFv, the variable region of heavy chain (V_{H}) of the scFv, Fc domain, may be fused directly (for example, by a peptide bond formed by a terminal carboxy group and an amino group) or through various linkers known in the art, particularly peptide linkers comprising one or more amino acids, typically about 2-20 amino acids. Suitable, nonimmunogenic peptide linkers include, for example, (GxSy)n, wherein the x and y are individually any integer selected from 1-10, preferably 2-8, 2-7, 2-6, 2-5, 2-4, and n is any integer selected from 1-10, preferably 2-8, 2-7, 2-6, 2-5, 2-4. In particular embodiments, in the dual targeting antigen binding molecule of the present invention, the second antigen binding moiety comprises a first Fab fused at the N-terminus of its Fab heavy chain to the C-terminus of the scFv, or at the C-terminus of its Fab heavy chain to the N-terminus of the scFv, by a peptide linker having the formula of (GxSy)n, wherein the x and y are individually any integer selected from 1-10, preferably 2-8, 2-7, 2-6, 2-5, 2-4, and n is any integer selected from 1-10, preferably 2-8, 2-7, 2-6, 2-5, 2-4. In particular embodiments, in the dual targeting antigen binding molecule of the present invention, the variable region of heavy chain (V_{H}) of the scFv is connected to the variable region of light chain (V_{L}) by a peptide linker having the formula of (GxSy)n, wherein the x and y are individually any integer selected from 1-10, preferably 2-8, 2-7, 2-6, 2-5, 2-4, and n is any integer selected from 1-10, preferably 2-8, 2-7, 2-6, 2-5, 2-4.

In certain embodiments said first and/or second antigen binding moeity is linked via a hinge region, or a part of a hinge region, to the Fc-domain. In certain embodiments said first and/or second antigen binding moeity is linked to the Fc-domain via a peptide linker having the formula of (GxSy)n, wherein the x and y are individually any integer selected from 1-10, preferably 2-8, 2-7, 2-6, 2-5, 2-4, and n is any integer selected from 1-10, preferably 2-8, 2-7, 2-6, 2-5, 2-4.

In certain embodiments, in the dual targeting antigen binding molecule of the present invention, the Fc domain is a human IgG Fc domain, preferably, a human IgG1 or IgG4 Fc domain. In certain embodiments, in the dual targeting antigen binding molecule of the present invention, the Fc domain comprises one or more modifications promoting the association of the first and the second subunit of the Fc domain. In preferred embodiments, in the CH3 domain of one subunit of the Fc domain, an amino acid residue is replaced with an amino acid residue having a larger side chain volume, thereby generating a protuberance within the CH3 domain of the subunit, and in the CH3 domain of the other subunit of the Fc domain an amino acid residue is replaced with an amino acid residue having a smaller side chain volume, thereby generating a cavity within the CH3 domain of the subunit, wherein the protuberance is protrudable into the cavity. In preferable embodiments, in the CH3 domain of one subunit of the Fc domain, the T366 residue is replaced with an amino acid residue having a larger side chain volume. In more preferable embodiments, in the CH3 domain of the one subunit of the Fc domain, one or more residues selected from T366, L368, and Y407 are replaced with one or more amino acid residues having a smaller side chain volume. In furthermore preferable embodiments, the Fc domain comprises a substitution of T366W in one subunit, and T366S, L368A and/or Y407V substitutions in the other subunit of the Fc domain.

In certain embodiments, in the dual targeting antigen binding molecule of the present invention, the Fc domain exhibits reduced binding affinity to an Fc receptor and/or reduced effector function, as compared to a native IgG1 or IgG4 Fc domain.

In certain embodiments, in the dual targeting antigen binding molecule of the present invention, the Fc domain comprises one or more amino acid substitutions that reduce binding to an Fc receptor and/or effector function, preferably, said one or more amino acid substitutions are at one or more positions selected from the group of L/F234, L235, D265, N297 and P329. More preferably, each subunit of the Fc domain comprises two amino acid substitutions that reduce binding to an activating Fc receptor and/or effector function wherein said amino acid substitutions are L/F234A and L235A.

In certain embodiments, in the dual targeting antigen binding molecule of the present invention, the Fc receptor is an Fcγ receptor, and the effector function is antibody-dependent cell-mediated cytotoxicity (ADCC), antibody-dependent cell-mediated phagocytosis (ADCP), or complement-dependent cytotoxicity (CDC).

In certain embodiments, the dual targeting antigen binding molecule of the present invention comprises an amino acid substitution at the position of S228 of IgG, preferably, the amino acid substitution at the position of S228 is S228P.

In certain embodiments, the first Fab and the second Fab are both anti-CD20 Fab. In certain embodiments, the first Fab and the second Fab comprise one, two, three, four, five or six CDRs selected from SEQ ID NO:3, 4, 5, 8, 9 and 10. In certain embodiments, the anti-CD3 scFV comprise one, two, three, four, five or six CDRs selected from SEQ ID NO:13, 14, 15, 18, 19 and 20. In certain embodiments, the first Fab and the second Fab are identical and comprise six CDRs selected from SEQ ID NO:3, 4, 5, 8, 9 and 10.

In certain embodiments, the dual targeting antigen binding molecule of the present invention comprises the first Fab and the second Fab comprising six CDRs selected from SEQ ID NO:3, 4, 5, 8, 9 and 10, and the anti-CD3 scFV comprising the six CDRs selected from SEQ ID NO:13, 14, 15, 18, 19 and 20.

In certain embodiments, the first Fab and the second Fab comprise variable regions of heavy chain and light chain comprising amino acid sequences shown by SEQ ID NO:2 and SEQ ID NO:7 respectively, or comprising amino acid sequences having at least 80%, 81%, 82%, 83%, 84%, 85%, 86%, 87%, 88%, 89%, 90%, 91%, 92%, 93%, 94%, 95%, 96%, 97%, 98%, 99% identity with SEQ ID NO:2 and SEQ ID NO:7 respectively. In certain embodiments, the first Fab and the second Fab are identical and comprise variable regions of heavy chain and light chain as shown by SEQ ID NO:2 and SEQ ID NO:7 respectively. In certain embodiments, the anti-CD3 scFV comprise variable regions of heavy chain and light chain comprising amino acid sequences as shown by SEQ ID NO:12 and SEQ ID NO:17 respectively, or comprising amino acid sequences having at least 80%, 81%, 82%, 83%, 84%, 85%, 86%, 87%, 88%, 89%, 90%, 91%, 92%, 93%, 94%, 95%, 96%, 97%, 98%, 99% identity with SEQ ID NO:12 and SEQ ID NO:17 respectively. In certain embodiments, the anti-CD3 scFV comprise variable regions of heavy chain and light chain comprising amino acid sequences as shown by SEQ ID NO:22 and SEQ ID NO:17 respectively, or comprising amino acid sequences having at least 80%, 81%, 82%, 83%, 84%, 85%, 86%, 87%, 88%, 89%, 90%, 91%, 92%, 93%, 94%, 95%, 96%, 97%, 98%, 99% identity with SEQ ID NO:22 and SEQ ID NO:17 respectively.

In certain embodiments, the dual targeting antigen binding molecule of the present invention comprises the first Fab and the second Fab comprising variable regions of heavy chain and light chain as shown by SEQ ID NO:2 and SEQ ID NO:7 respectively and the anti-CD3 scFV comprising the variable regions of heavy chain and light chain as shown by SEQ ID NO:12 and SEQ ID NO:17 respectively. In certain embodiments, the dual targeting antigen binding molecule of the present invention comprises the first Fab and the second Fab comprising variable regions of heavy chain and light chain as shown by SEQ ID NO:2 and SEQ ID NO:7 respectively and the anti-CD3 scFV comprising the variable regions of heavy chain and light chain as shown by SEQ ID NO:22 and SEQ ID NO:17 respectively.

In one aspect, the present invention is related to a dual targeting antigen binding molecule, comprising a) an Fc domain of human IgG, consisting of a first and a second subunit capable of stable association, b) a first antigen binding moiety capable of specific binding to a T cell-activating antigen, comprising a scFv, and c) a second antigen binding moiety capable of specific binding to a target cell antigen, comprising a first Fab and a second Fab, wherein

1) the scFv, at the N-terminus of the variable region of heavy chain (V_{H}) of the scFv or at the N-terminus of the variable region of light chain (V_{L}) of the scFv, is fused to the C-terminus of the Fab heavy chain of the first Fab, and at the C-terminus of the variable region of heavy chain (V_{H}) or the variable region of light chain (V_{L}) of the scFv, is fused to the first subunit of the Fc domain comprising a substitution of T366 with an amino acid residue having a larger side chain,

2) the second Fab, at the C-terminus of the Fab heavy chain, is fused to the second subunit of the Fc domain comprising one or more substitutions of T366, L368, and/or Y407 with an amino acid residue having a smaller side chain volume.

In preferable embodiments, in the dual targeting antigen binding molecule of the present invention, the Fc domain comprises a substitution of T366W in the first subunit, and T366S, L368A and Y407V substitutions in the second subunit of the Fc domain. In more preferable embodiments, the Fc domain further comprises one or more amino acid substitutions that reduce the binding to an Fc receptor and/or the effector function. In furthermore preferable embodiments, said one or more amino acid substitutions are at one or more positions selected from the group of L/F234, L235, D265, N297 and P329. In most preferable embodiments, each subunit of the Fc domain comprises two amino acid substitutions that reduce the binding to an activating Fc receptor and/or the effector function, wherein the amino acid substitutions are L/F234A and L235A.

In certain embodiments, the dual targeting antigen binding molecule of the present invention further comprises an amino acid substitution at the position of 5228 of IgG4, preferable, 5228P.

In certain embodiments, in the dual targeting antigen binding molecule of the present invention, the scFv is fused to the Fab heavy chain of the first Fab by a peptide linker, preferably, (GxSy)n, wherein the x and y is individually any integer selected from 1 to 5, and n is any integer selected from 1-5.

A skilled person in the art can understand that there may be a linker between the variable region of heavy chain (V_{H}) and the variable region of light chain (V_{L}) of the scFv comprised in the dual targeting antigen binding molecule of the present invention. The linker may be a peptide linker, preferably, (GxSy)n, wherein the x and y are individually any integer selected from 1-5, and n is any integer selected from 1-5.

In one aspect, also contemplated by the present invention is a dual targeting antigen binding molecule, comprising a) an Fc domain of human IgG, consisting of a first and a second subunit capable of stable association, b) a first antigen binding moiety capable of specific binding to a T cell-activating antigen, comprising a scFv, and c) a second antigen binding moiety capable of specific binding to a target cell antigen, comprising a first Fab and a second Fab, wherein

1) the scFv, at the C-terminus of variable region of heavy chain (V_{H}) of the scFv or at the C-terminus of variable region of light chain (V_{L}) of the scFv, is fused to the N-terminus of the Fab heavy chain of the first Fab, and the first Fab, at the C-terminus of the Fab heavy chain, is fused to the first subunit of the Fc domain comprising a substitution of T366 with an amino acid residue having a larger side chain,

2) the second Fab, at the C-terminus of the Fab heavy chain, is fused to the second subunit of the Fc domain comprising one or more substitutions of the residues T366, L368, and/or Y407 with an amino acid residue having a smaller side chain volume. Preferably, the Fc domain comprises a substitution of T366W in the first subunit, and T366S, L368A and Y407V substitutions in the second subunit of the Fc domain. More preferably, the Fc domain further comprises one or more amino acid substitutions that reduce the binding to an Fc receptor and/or the effector function. Furthermore preferably, the one or more amino acid substitutions are at one or more positions selected from the group of L/F234, L235, D265, N297 and P329. Most preferably, each subunit of the Fc domain comprises two amino acid substitutions that reduce the binding to an activating Fc receptor and/or effector function, wherein said amino acid substitutions are L/F234A and L235A.

In certain embodiments, the dual targeting antigen binding molecule of the present invention comprise a substitution at the position of S228 of IgG4, preferably, S228P.

In certain embodiments, in the dual targeting antigen binding molecule of the present invention, the scFv is fused to the Fab heavy chain of the first Fab by a peptide linker, preferably, (GxSy)n, wherein the x and y is individually any integer selected from 1 to 5, and n is any integer selected from 1-5.

In certain embodiments, in the dual targeting antigen binding molecule of the present invention, the first subunit and said second subunit of the Fc domain have been modified to comprise one or more charged amino acids electrostatically favorable for heterodimer formation. Preferably, the first subunit of the Fc domain comprises amino acid mutations E356K, E357K and/or D399K and said second subunit comprises amino acid mutations K370E, K409E and/or K439E. More preferably, the first subunit of the Fc domain comprises amino acid mutations K392D and K409D and the second subunit of the Fc domain comprises amino acid mutations E356K and D399K (DDKK).

In certain embodiments, in the dual targeting antigen binding molecule of the present invention, the T cell-activating antigen is any one selected from the group consisting of CD3, 4-1BB, PD-1 and CD40L/CD154.

In certain embodiments, in the dual targeting antigen binding molecule of the present invention, the target cell antigen is a tumor-specific antigen (TSA) or tumor-associated antigen (TAA). Preferably, the target cell antigen is any one selected from the group consisting of: CD19, CD20, CD33, CD38, Melanoma-associated Chondroitin Sulfate Proteoglycan (MCSP), cell surface associated mucin 1 (MUC1), Epidermal Growth Factor Receptor (EGFR), HER2, Carcinoembryonic Antigen (CEA), B7-H1, B7-H3, B7-H4, Glypican-3, Mesothelin, Trophoblast glycoprotein (5T4), Transferrin receptor 1 (TfR1) and Fibroblast Activation Protein (FAP).

In certain embodiments, the dual targeting antigen binding molecule of the present invention is a T cell redirecting bispecific antigen binding antibody, or a fragment thereof capable of specific binding to a T cell-activating antigen and a target cell antigen.

In one aspect, the presentation is involved in an isolated polynucleotide encoding the dual targeting antigen binding molecule of the present invention, a polypeptide encoded by the isolated polynucleotide, a vector comprising the isolated polynucleotide, or a host cell comprising said isolated polynucleotide or said vector.

In one aspect, the presentation contemplates a method of producing the dual targeting antigen binding molecule of the present invention, comprising the steps of a) culturing the host cell under conditions suitable for the expression of the dual targeting antigen binding molecule and b) recovering the dual targeting antigen binding molecule. Meanwhile, the present invention is also extended to the dual targeting antigen binding molecule produced by the method of the present invention.

In one aspect, the presentation covers a pharmaceutical composition comprising the dual targeting antigen binding molecule of the present invention and a pharmaceutically acceptable carrier, an article of manufacture or kit comprising the dual targeting antigen binding molecule or the pharmaceutical composition of the present invention in a container and an instruction indicating how to use the dual targeting antigen binding molecule.

In one aspect, the present invention also discloses the use of the dual targeting antigen binding molecule or the pharmaceutical composition of the present invention, for example, the use for the treatment of different kinds of cancers.

In one aspect, the subject matter also covered by the present invention is the use of the dual targeting antigen binding molecule for the manufacture of a medicament for the treatment of a disease in an individual in need thereof.

In one aspect, the present invention is related to a method of treating a disease in an individual, especially a cancer, comprising administering to said individual a therapeutically effective amount of the dual targeting antigen binding molecule or the pharmaceutical composition of the present invention.

In one aspect, the present invention is related to a method for inducing lysis of target cells, comprising contacting target cells with the dual targeting antigen binding molecule of the present invention in the presence of T cells.

In the above stated embodiments, the dual targeting antigen binding molecule of the present invention is preferably a T cell redirecting bispecific antigen binding antibody or a fragment thereof, capable of specific binding to a T cell-activating antigen and a target cell antigen. The cell-activating antigen may be any one selected from the group consisting of CD3, 4-1BB, PD-1 and CD40L/CD154, and the target cell antigen may be a tumor-specific antigen (TSA) or tumor-associated antigen (TAA). Preferably, the target cell antigen is any one selected from the group consisting of: CD19, CD20, CD33, CD38, Melanoma-associated Chondroitin Sulfate Proteoglycan (MCSP), cell surface associated mucin 1 (MUC1), Epidermal Growth Factor Receptor (EGFR), HER2, Carcinoembryonic Antigen (CEA), B7-H1, B7-H3, B7-H4, Glypican-3, Mesothelin, Trophoblast glycoprotein (5T4), Transferrin receptor 1 (TfR1) and Fibroblast Activation Protein (FAP).

### BRIEF DESCRIPTION OF THE DRAWING

FIG. 1A-B. Schematic design of the bispecific T Cell-Redirecting Antibodies (TRAB) structure. The bispecific antibodies comprise a first antigen binding moiety capable of specific binding to a T cell-activating antigen, a second antigen binding moiety capable of specific binding to a target cell antigen (TSA or TAA), and a Fc domain consisting of a first and a second subunit, wherein the first antigen binding moiety comprises a scFv and the second antigen binding moiety comprises a first Fab and a second Fab. For illustration, the schematic design takes CD3 as an example of the T cell-activating antigen, and TAA stands for the target cell antigen. The illustrated bispecific antibodies are designated as TAA×CD3 SimBody™ and SomBody™.

FIG. 2A-B. Structures of the Molecule A and Molecule B CD20×CD3 (Fab-scFv)₂-Fc fusion proteins.

FIG. 3A-B. Structures of two CD20×CD3 SimBody™ TRABs.

FIG. 4A-B. Structures of the Molecule C and Molecule D CD20×CD3 (scFv-Fab)₂-Fc fusion proteins.

FIG. 5A-B. Structures of two CD20×CD3 SomBody™ TRABs.

FIG. 6. Schematic diagrams of constructed plasmids for making the CD20×CD3 SimBody™ or CD20×CD3 SomBody™ TRABs.

FIG. 7A-B. SDS-PAGE analysis of the test articles after protein A purification.

FIG. 8. SDS-PAGE analysis of CD20×CD3 SimBody™-A after cation exchange.

FIG. 9. SDS-PAGE analysis of CD20×CD3 SimBody™-B after cation exchange.

FIG. 10. SDS-PAGE analysis of CD20×CD3 SomBody™-C after cation exchange.

FIG. 11. SDS-PAGE analysis of CD20×CD3 SomBody™-D after cation exchange.

FIG. 12A-H. SEC-HPLC analysis of CD20×CD3 SimBody™ or SomBody™ test articles.

FIG. 13A-H. NR-CE-SDS analysis of CD20×CD3 SimBody™ or SomBody™ test articles.

FIG. 14A-H. R-CE-SDS analysis of CD20×CD3 SimBody™ or SomBody™ test articles.

FIG. 15. The binding curves of CD20×CD3 SimBodies to CD20-positive Raji cells.

FIG. 16. The binding curves of CD20×CD3 SomBodies to CD20-positive Raji cells.

FIG. 17. The binding curves of CD20×CD3 SimBodies to CD3-positive Jurkat cells.

FIG. 18. The binding curves of CD20×CD3 SomBodies to CD3-positive Jurkat cells.

FIG. 19. CD20×CD3 SimBody™ redirected T cells from human PBMC to lyse human lymphoma B cell line Daudi in a concentration-dependent manner.

FIG. 20A-D. Early and late T cell activation by CD20×CD3 SimBodies.

FIG. 21. Dosing and sample collection regimen of the in vivo B cell depletion study.

FIG. 22. CD19+ B cell depletion percentages of the in vivo study

FIG. 23. CD4+ T cell percentage changes of the in vivo study

FIG. 24. CD8+ T cell percentage changes of the in vivo study

FIG. 25. Mass spectrometry analysis of total molecular weight of CD20×CD3 SimBody™-A.

FIG. 26. Mass spectrometry analysis of the molecular weight of CD20×CD3 SimBody™-A light chain.

FIG. 27A-B. Mass spectrometry analysis of the molecular weight of CD20×CD3 SimBody™-A heavy chain 1 and heavy chain 2.

### DETAILED DESCRIPTION OF THE INVENTION

The present invention is related to dual targeting antigen binding molecules, especially, bispecific T Cell-Redirecting Antibodies (TRAB) comprising two different antigen binding components, one for specific binding to a T cell-activating antigen, and the other for specific binding to a target cell antigen, for example a tumor-specific antigen (TSA) or tumor-associated antigen (TAA). By specific binding to the T cell-activating antigen and the target cell antigen, the bispecific molecules (antibodies) redirect T cells to the spot where the target cells including cancer cells are located, and the target cells are destroyed by the activated T cells and/or other effector cells by antibody-dependent cell-mediated cytotoxicity (ADCC), antibody-dependent cell-mediated phagocytosis (ADCP) or complement-dependent cytotoxicity (CDC).

### Definition

As used in the present invention, “dual targeting antigen binding molecule” means the molecule is able to target and bind not only a T cell-activating antigen but also a target cell antigen. The dual targeting antigen binding molecule includes, for example, an antibody, an antibody fragment and a polypeptide dually targeting and binding antigens, for example, CD3 molecule and any TSA or TAA antigen. The molecule can be presented as an assembled antibody or a polymeric polypeptide molecule assembled by different portions derived from an antibody, for example, CDR domain, variable region, CH1, CH2 and/or CH3 domain, Fv, scFv and Fab fragment and/or Fc domain. The assembled antibody and polypeptide molecule specifically bind to antigens for example, CD3 molecule and any TSA or TAA antigen.

The term “antibody (Ab) or antibodies (Abs)” of the present invention covers antibodies with structural characteristics of a native antibody and antibody-like molecules having structural characteristics different from a native antibody but exhibiting binding specificity to one or more specific antigens. The term antibodies is intended to encompass immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The terms “heavy chain”, “light chain”, “light chain variable region” (“V_{L}”), “heavy chain variable region” (“V_{H}”), “framework region” (“FR”), “heavy chain constant domain (“CH”), “light chain constant domain (“CL”) refer to domains in naturally occurring immunoglobulins and the corresponding domains of synthetic (e.g., recombinant) binding proteins (e.g., humanized antibodies). The basic structural unit of naturally occurring immunoglobulins (e.g., IgG) is a tetramer having two light chains and two heavy chains. The amino-terminal (“N”) portion of each chain includes a variable region of about 100 to 110 or more amino acids primarily responsible for antigen recognition. The carboxy-terminal (“C”) portion of each chain defines a constant region, with light chains having a single constant domain and heavy chains usually having three constant domains and a hinge region. Thus, the structure of the light chains of a naturally occurring IgG molecule is N-V_{L}-CL-C and the structure of IgG heavy chains is N-V_{H}-CH1-H-CH2-CH3-C (where H is the hinge region). The variable regions of an IgG molecule comprise the complementarity determining regions (CDRs), which contain the residues in contact with antigen and non-CDR segments, referred to as framework segments, which maintain the structure and determine the positioning of the CDR loops. Thus, the V_{L} and V_{H} domains have the structure N-FR1-CDR1-FR2-CDR2-FR3-CDR3-FR4-C.

As used herein the phrase “bispecific antibody” or “bispecific antigen binding antibody” designates antibodies (as hereinabove defined) having two binding specificities, one of which is for specifically binding with a T cell-activating antigen, for example, CD3, 4-1BB, PD-1 or CD40L/CD154, and the other is for specifically binding with a target cell antigen, for example, a tumor-specific antigen (TSA) or tumor-associated antigen (TAA), such as CD19, CD20, CD33, CD38, Melanoma-associated Chondroitin Sulfate Proteoglycan (MCSP), cell surface associated mucin 1 (MUC1), Epidermal Growth Factor Receptor (EGFR), HER2, Carcinoembryonic Antigen (CEA), B7-H1, B7-H3, B7-H4, Glypican-3, Mesothelin, Trophoblast glycoprotein (5T4), Transferrin receptor 1 (TfR1) and Fibroblast Activation Protein (FAP).

A native antibody is usually heterotetrameric glycoprotein, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy or light chain also has regularly spaced intrachain disulfide bridges. Each heavy chain has at one end a variable region (V_{H}) followed by several constant regions. Each light chain has a variable region (V_{L}) at one end and a constant region at its other end; the constant domain of the light chain is aligned with the first constant region of the heavy chain, and the light chain variable region (V_{L}) is aligned with the variable domain of the heavy chain (V_{H}).

In the native antibody, the variability is not evenly distributed through the variable regions of antibodies. It is concentrated in three segments called complementarity determining regions (CDRs) or hypervariable regions both in the light chain and the heavy chain variable regions. The more highly conserved portions of variable domains are called the framework (FR). The variable regions of native heavy and light chains each comprise four FR regions, connected by three CDRs. The CDRs in each chain are held together in proximity with the FR regions and, with the CDRs from the other chain, contribute to the formation of the antigen binding site of antibodies [see Kabat, E. A. et al., Sequences of Proteins of Immunological Interest National Institute of Health, Bethesda, Md. (1987)]. The constant regions are not involved directly in binding an antibody to an antigen, but exhibit various effector functions, such as participation of the antibody in antibody-dependent cellular toxicity (ADCC).

Antibodies as used herein can be intact antibody molecules, or “antibody fragments”. “Antibody fragments” as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, Fv and scFv fragments.

Papain digestion of antibodies produces two identical antigen binding fragments, called “Fab” fragments, each with a single antigen binding site, and a residual “Fc” fragment, whose name reflects its ability to crystallize readily. The “Fab” fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation for Fab′ in which the cysteine residue(s) of the constant domains have a free thiol group. F(ab′) fragments are produced by cleavage of the disulfide bond at the hinge cysteines of the F(ab′)₂ which is pepsin digestion product.

“Fv” fragments are antibody fragments which contain a complete antigen recognition and binding site, consisting of a dimer of one heavy and one light chain variable region in a tight, non-covalent association, while “Single-chain Fv (scFv)” fragments consist of one heavy- and one light-chain variable region covalently linked by a flexible peptide linker in one single polypeptide chain. It is in this configuration that the three CDRs of each variable region of heavy- and light chain interact to define an antigen-binding site on the surface of the V_{H}-V_{L} dimer. Collectively, the six CDRs confer antigen-binding specificity to the antibody.

In certain embodiments of the present invention, the dual targeting antigen binding molecule (including T cell redirecting bispecific antigen binding antibody) comprises a first antigen binding moiety comprising a scFv, wherein the scFv comprises a variable region of heavy chain (V_{H}) and a variable region of light chain (V_{L}) from the N-terminus to C-terminus of the scFv, or a variable region of light chain (V_{L}) and a variable region of heavy chain (V_{H}) from the N-terminus to C-terminus of the scFv. In preferable embodiments, the scFv can be Anti-CD3 scFv, Anti-4-1BB scFv, or Anti-CD40L/CD154 scFv, derived from any Anti-CD3 antibody, Anti-4-1BB antibody, or Anti-CD40L/CD154 antibody. In certain embodiments of the present invention, the dual targeting antigen binding molecule (T cell redirecting bispecific antigen binding antibody) comprises a second antigen binding moiety comprising a first Fab fused at the C-terminus of the Fab heavy chain to the scFv and a second Fab fused at the C-terminus of the Fab heavy chain to the Fc domain. In preferable embodiments, the first Fab and second Fab are identical and specific binding to a TSA and TAA antigen (Anti-TSA Fab or Anti-TAA Fab). Fab fragments can be derived from any antibody against any antigen selected from the group consisting of CD19, CD20, CD33, CD38, Melanoma-associated Chondroitin Sulfate Proteoglycan (MCSP), cell surface associated mucin 1 (MUC1), Epidermal Growth Factor Receptor (EGFR), HER2, Carcinoembryonic Antigen (CEA), B7-H1, B7-H3, B7-H4, Glypican-3, Mesothelin, Trophoblast glycoprotein (5T4), Transferrin receptor 1 (TfR1) and Fibroblast Activation Protein (FAP).

As used herein, the term “antigen binding moiety” refers to a polypeptide that specifically binds to an antigen. In the present invention, the first antigen binding moiety and the second antigen binding moiety bind to at least two distinct antigens. For example, the first antigen binding moiety binds to a T cell activating antigen, and the second antigen binding moiety binds to a target cell antigen, for example a protein expressed by cancer cells. In structure, antigen binding moieties comprise fragments from antibodies, for example, Fab and scFv fragments, linked by a peptide linker.

In certain embodiments, the dual targeting antigen binding molecule (T cell redirecting bispecific antigen binding antibody) of the present invention comprises a Fc domain comprising a first subunit and a second subunit capable of stable association.

“Fc domain” can also be called “Fc region”, means fragment crystallizable domain is the tail region of an antibody that interacts with cell surface receptors called Fc receptors and some proteins of the complement system. In IgG, IgA and IgD antibody isotypes, the Fc domain (region) is composed of two identical subunits (first subunit and second subunit), with each consisting of CH2 and CH3 constant domains derived from heavy chain of antibody; IgM and IgE Fc domain (region), is composed of two identical subunits (first subunit and second subunit), with each consisting of CH2, CH3 and CH4 constant domains derived from heavy chain of antibody. The Fc domain binds to various cell receptors and complement proteins. In this way, it mediates different physiological effects of antibodies.

Fc domain (region) is positioned at the C-terminal region of a heavy chain of an antibody. Although the boundaries may vary slightly, the human IgG heavy chain Fc region is defined to stretch from Cys226 to the carboxy terminus. The Fc region of an IgG comprises two constant domains, CH2 and CH3. The CH2 domain of a human IgG Fc region (also referred to as “Cy2” domain) usually extends from amino acid 231 to amino acid 338, and the CH3 domain of a human IgG Fc region usually extends from amino acids 342 to 447.

The term “hinge region” is generally defined as stretching from Glu216 to Pro230 of human IgG1. Hinge regions of other IgG isotypes may be aligned with the IgG1 sequence by placing the first and last cysteine residues forming inter-heavy chain S-S bonds in the same positions. The Fc domain, as stated hereinabove, is from a human IgG, preferably, a human IgG1 or IgG4, preferably, comprising one or more modifications promoting the association of the first and the second subunit of the Fc domain, for example, by generating knob-into-hole structure to strength the association. Such knob-into-hole structure can be produced by replacing an amino acid residue in the CH3 domain of the first subunit of the Fc domain with an amino acid residue having a larger side chain volume, thereby generating a protuberance (knob) within the CH3 domain of the first subunit, and replacing an amino acid in the CH3 domain of the second subunit of the Fc domain with an amino acid residue having a smaller side chain volume, thereby generating a cavity (hole) within the CH3 domain of the second subunit, wherein the protuberance is protrudable into the cavity to promote the stable association of the first and second subunits of the Fc domain.

Altering the Fc domain may promote the generation of heavy chain heterodimers, resulting in bispecific antibodies comprising two different heavy-light chain pairs. To facilitate the formation of heterodimers the interface between a pair of Fc subunits is engineered to maximize the percentage of heterodimers by, for example, introducing the knob-into-hole structure, as hereinabove mentioned. This provides a mechanism for increasing the yield of the heterodimer over other unwanted end-products such as homodimers. CH3 modifications include, for example, Y407V/T366S/L368A on one heavy chain and T366W on the other heavy chain; S354C/T366W on one heavy chain and Y349C/Y407V/T366S/L368A on the other heavy chain. Additional modifications resulting in a protrusion (knob) on one chain and a cavity (hole) on the other are described in U.S. Pat. No. 7,183,076; and Merchant et al, 1998, Nat. Biotech 16:677-681. Other modifications which may be used to generate heterodimers include but are not limited to those which alter the charge polarity across the Fc dimer interface such that co-expression of electrostatically matched Fc subunits results in heterodimerization. Modifications which alter the charge polarity include, but are not limited to:

K370E/D399K/K439D D356K/E357K/K409D

K409D D399K

K409E D399K

K409E D399R

K409D D399R

D339K E356K

D399K/E356K K409D/K392D

D399K/E356K K409D/K439D D399K/E357K K409D/K370D

D399K/E356K/E357K K409D/K392D/K370D

D399K/E357K K409D/K392D

K392D/K409D D399K

K409D/K360D D399K.

They are also disclosed in WO 2007/147901; Gunasekaran et al, 2010, JBC 285: 19637-46. In addition, Davis et al. (2010, Prot. Eng. Design & Selection 23: 195-202) describe a heterodimeric Fc platform using strand-exchanged engineered domain (SEED) CH3 regions which are derivatives of human IgG and IgA CH3 domains (also, see WO 2007/1 10205).

Other modifications and/or substitutions and/or additions and/or deletions of the Fc domain will be apparent to one skilled in the art to achieve stable association and/or promote heterodimer formation. These Fc variants disclosed in the art may be combined with the Fc domain disclosed by the present invention and those documents disclosed the Fc variants are incorporated into this application in their entirety as reference.

A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

When referring to antibodies, the assignment of amino acids to each domain is in accordance with Kabat, Sequences of Proteins of Immunological Interest (National Institutes of Health, Bethesda, Md., 1987 and 1991), which is expressly incorporated herein by reference. Throughout the present specification, the numbering of the residues in an IgG heavy chain is that of the EU index as in Kabat, and refers to the numbering of the human IgG1 EU antibody.

As used herein, the term “cancer” refers to a neoplasm or tumor resulting from abnormal uncontrolled growth of cells. As used herein, cancer explicitly includes, leukemias and lymphomas. In some embodiments, cancer refers to a benign tumor, which has remained localized. In other embodiments, cancer refers to a malignant tumor, which has invaded and destroyed neighboring body structures and spread to distant sites. In some embodiments, the cancer is associated with a specific cancer antigen.

The present invention will be described with respect to particular embodiments and with reference to certain drawings, but the invention is not limited thereto but only by the claims. The term “comprising” as used in the present description and claims does not exclude other elements or steps. Where an indefinite or definite article is used when referring to a singular noun e.g. “a” or “an”, “the”, this includes a plural of that noun unless something else is specifically stated.

Terms or definitions have the same meaning as they would to one skilled in the art of the present invention, unless specifically defined herein. As to terms and methods, such as polypeptide, polynucleotide, vector, host cell, cloning, transfection, transduction, expression, etc., commonly used in genetically engineering techniques, practitioners may particularly refer to, for example, Sambrook et al., Molecular Cloning: A Laboratory Manual, 2nd ed., Cold Spring Harbor Press, Plainsview, N.Y. (1989); and Ausubel et al., Current Protocols in Molecular Biology (Supplement 47), John Wiley & Sons, New York (1999).

### The Dual Targeting Antigen Binding Molecule of the Invention

The present invention is related to a dual targeting antigen binding molecule, comprising a first antigen binding moiety capable of specific binding to a T cell-activating antigen, and a second antigen binding moiety capable of specific binding to a target cell antigen, wherein the first antigen binding moiety comprises a scFv and the second antigen binding moiety comprises a first Fab and a second Fab. In preferable embodiments, the dual targeting antigen binding molecule further comprises an Fc domain consisting of a first and a second subunit capable of stable association. In preferable embodiments, the dual targeting antigen binding molecule of the present invention is a T cell redirecting bispecific antigen binding antibody, or a fragment thereof capable of specific binding to a T cell-activating antigen and a target cell antigen.

In one aspect, the present invention is related to a dual targeting antigen binding molecule, comprising a first antigen binding moiety capable of specific binding to a T cell-activating antigen, and a second antigen binding moiety capable of specific binding to a target cell antigen. In certain embodiments, the first antigen binding moiety comprises a scFv and the second antigen binding moiety comprises a first Fab and a second Fab. In certain embodiments, the variable region of light chain (V_{L}) and a variable region of heavy chain (V_{H}) in the scFv can be reverted in direction. In certain embodiments, the first Fab fused at the C-terminus of the Fab heavy chain to the scFv fused to the Fc domain; and the second Fab is fused at the C-terminus of the Fab heavy chain to the Fc domain. Thus, the structure of one polypeptide of the assembled dual targeting antigen binding molecule can be presented as N-V_{H} (the First Fab)-V_{L}(scFv)-V_{H}(scFv)-Fc or N-V_{H} (the First Fab)-V_{H}(scFv)-V_{L}(scFv)-Fc, and the structure of the other polypeptide of the assembled dual targeting antigen binding molecule can be presented as N-V_{H} (the Second Fab)-Fc.

In certain embodiments, the first Fab fused at its N-terminus of the Fab heavy chain to the C-terminus of the variable region of heavy chain (V_{H}) of the scFv; and the second Fab is fused at the C-terminus of the Fab heavy chain to the Fc domain. In certain embodiments, the first Fab fused at its N-terminus of the Fab heavy chain to the C-terminus of the variable region of light chain (V_{L}) of the scFv. In certain embodiments, the variable region of light chain (V_{L}) and a variable region of heavy chain (V_{H}) in the scFv can be reverted in direction. Thus, the structure of one polypeptide of the assembled dual targeting antigen binding molecule may be presented as N-V_{L}(scFv)-V_{H}(scFv)-V_{H} (the First Fab)-Fc or N-V_{H}(scFv)-V_{L}(scFv)-V_{H} (the First Fab)-Fc, and the structure of the other polypeptide of the assembled dual targeting antigen binding molecule can be presented as N-V_{H} (the Second Fab)-Fc.

For assembling the dual targeting antigen binding molecule of the present invention, the portions, such as CDRs, FRs, V_{H}, V_{L}, scFv, Fab, CH1, CH2 and CH3, derived from an antibody can be fused to each other by a linker, preferably a peptide linker (GxSy)n described herein, or by a covalent bond, for example, peptide bond form by terminal carboxy and amino groups.

The dual targeting antigen binding molecule of the present invention specifically binds to a T cell-activating antigen and a target cell antigen in view of the first antigen binding moiety and the second antigen binding moiety. By “specific binding”, it is meant that the binding is selective for the antigen and can be discriminated from unwanted or non-specific interactions. The ability of specific binding can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR.

The capability of an antigen binding molecule or an antibody binding to the cognate antigen can be determined by “Affinity”, which refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair. The affinity can generally be represented by the dissociation constant (KD), which is the ratio of dissociation and association rate constants (koff and kon, respectively). Thus, equivalent affinities may comprise different rate constants, as long as the ratio of the rate constants remains the same. Affinity can be measured by well-established methods known in the art, including Surface Plasmon Resonance (SPR).

The dual targeting antigen binding molecule of the present invention, in further preferable embodiments, comprises “Fc domain” or “Fc region” at the C-terminal region of an immunoglobulin heavy chain that contains at least a portion of the constant region. For example, an IgG CH2 and an IgG CH3 may form a subunit, and the Fc domain of the antigen binding molecule or antibody described herein may comprise a first subunit and second subunit of an IgG Fc domain, and further comprises modifications promoting the association of the first and the second subunit of the Fc domain and reducing or preventing the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain. In one embodiment a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

For example, one subunit of dual targeting antigen binding molecule of the present invention comprises a substitution of T366 with an amino acid residue having a larger side chain and the other subunit comprises one or more substitutions of the residues T366, L368, and/or Y407 with an amino acid residue having a smaller side chain volume. In certain embodiments, one subunit of the dual targeting antigen binding molecule of the present invention comprises amino acid mutations E356K, E357K and/or D399K and the other subunit comprises amino acid mutations K370E, K409E and/or K439E. In certain embodiments, one subunit of the dual targeting antigen binding molecule of the present invention comprises amino acid mutations K392D and K409D and the other subunit comprises amino acid mutations amino acid mutations E356K and D399K (DDKK).

In certain embodiments, the dual targeting antigen binding molecule of the present invention further comprises one or more amino acid substitutions that reduce the binding to an Fc receptor and/or the effector function, for example, one or more positions selected from the group of L/F234, L235, D265, N297 and P329. In certain embodiments, the dual targeting antigen binding molecule of the present invention further comprises a substitution at the position of S228 (preferably, S228P) of IgG4.

### Preparation of the Dual Targeting Antigen Binding Molecule of the Invention

The dual targeting antigen binding molecule of the present invention comprises a first antigen binding moiety capable of specific binding to a T cell-activating antigen, and a second antigen binding moiety capable of specific binding to a target cell antigen, wherein the first antigen binding moiety comprises a scFv and the second antigen binding moiety comprises a first Fab and a second Fab.

The scFv and Fab molecules may be any of the art, or any future scFv and Fab molecules. They may be derived from a naturally occurring antibody of any species including, but not limited to mouse, goat, rabbit, and human, or can be recombinant, CDR-grafted, humanized, and/or in vitro generated (e.g., selected by phage display). For example, a scFv and Fab molecule can be obtained by immunization of an animal with the desired antigen and subsequent isolation of the mRNA of an antibody fragment of interest, and by reverse transcription and polymerase chain reaction, a gene library of an antibody fragment of interest containing several million clones is produced. Screening techniques like phage display and ribosome display help to identify the clones binding the antigen. A different method uses gene libraries from animals that have not been immunized beforehand. Such naive libraries usually contain only antibodies with low affinity to the desired antigen, making it necessary to apply affinity maturation by random mutagenesis as an additional step. When the most potent clones have been identified, their DNA sequence is optimized, for example to improve their stability towards enzymes. Another goal is humanization to prevent immunological reactions of the human organism against the antibody. The final step is the translation of the optimized antibody fragment in E. coli, Saccharomyces cerevisiae or other suitable organisms.

In certain embodiments, the first Fab and the second Fab are both anti-CD20 Fab. In certain embodiments, the first Fab and the second Fab comprise one, two, three, four, five or six CDRs selected from SEQ ID NO:3, 4, 5, 8, 9 and 10. In certain embodiments, the anti-CD3 scFV comprise one, two, three, four, five or six CDRs selected from SEQ ID NO:13, 14, 15, 18, 19 and 20. In certain embodiments, the first Fab and the second Fab are identical and comprise six CDRs selected from SEQ ID NO:3, 4, 5, 8, 9 and 10.

In certain embodiments, the first Fab and the second Fab comprise variable regions of heavy chain and light chain comprising amino acid sequences shown by SEQ ID NO:2 and SEQ ID NO:7 respectively, or comprising amino acid sequences having at least 80%, 81%, 82%, 83%, 84%, 85%, 86%, 87%, 88%, 89%, 90%, 91%, 92%, 93%, 94%, 95%, 96%, 97%, 98%, 99% identity with SEQ ID NO:2 and SEQ ID NO:7 respectively. In certain embodiments, the anti-CD3 scFV comprise variable regions of heavy chain and light chain comprising amino acid sequences as shown by SEQ ID NO:12 and SEQ ID NO:17 respectively, or comprising amino acid sequences having at least 80%, 81%, 82%, 83%, 84%, 85%, 86%, 87%, 88%, 89%, 90%, 91%, 92%, 93%, 94%, 95%, 96%, 97%, 98%, 99% identity with SEQ ID NO:12 and SEQ ID NO:17 respectively. In certain embodiments, the anti-CD3 scFV comprise variable regions of heavy chain and light chain comprising amino acid sequences as shown by SEQ ID NO:22 and SEQ ID NO:17 respectively, or comprising amino acid sequences having at least 80%, 81%, 82%, 83%, 84%, 85%, 86%, 87%, 88%, 89%, 90%, 91%, 92%, 93%, 94%, 95%, 96%, 97%, 98%, 99% identity with SEQ ID NO:22 and SEQ ID NO:17 respectively.

In certain embodiments, the dual targeting antigen binding molecule of the present invention comprises the first Fab and the second Fab comprising variable regions of heavy chain and light chain as shown by SEQ ID NO:2 and SEQ ID NO:7 respectively and the anti-CD3 scFV comprising the variable regions of heavy chain and light chain as shown by SEQ ID NO:12 and SEQ ID NO:17 respectively. In certain embodiments, the dual targeting antigen binding molecule of the present invention comprises the first Fab and the second Fab comprising variable regions of heavy chain and light chain as shown by SEQ ID NO:2 and SEQ ID NO:7 respectively and the anti-CD3 scFV comprising the variable regions of heavy chain and light chain as shown by SEQ ID NO:22 and SEQ ID NO:17 respectively.

The dual targeting antigen binding molecule of the invention comprise different antigen binding moieties, and in one embodiment are fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of the dual targeting antigen binding molecule in recombinant production, it will thus be advantageous to introduce in the Fc domain of the dual targeting antigen binding molecule a modification promoting the association of the desired polypeptides.

Accordingly, in particular embodiments the Fc domain of the dual targeting antigen binding molecule of the invention comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain. In a specific embodiment said modification is a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. The protuberance and cavity can be made by altering the nucleic acid encoding the polypeptides, e.g. by site-specific mutagenesis, or by peptide synthesis.

In certain embodiments a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

In one aspect the invention provides a dual targeting antigen binding molecule, comprising a first antigen binding moiety capable of specific binding to a T cell-activating antigen, and a second antigen binding moiety capable of specific binding to a target cell antigen, and an Fc domain consisting of a first and a second subunit, wherein the first antigen binding moiety comprises a scFv and the second antigen binding moiety comprises a first Fab and a second Fab, and wherein the first subunit and said second subunit have been modified to comprise one or more charged amino acids electrostatically favorable to heterodimer formation.

The Fc domain confers to the dual targeting antigen binding molecule favorable pharmacokinetic properties, including a long serum half-life, but at the same time, it may lead to undesirable targeting of the dual targeting antigen binding molecule to cells expressing Fc receptors rather than to antigen-bearing target cells. Accordingly, in particular embodiments the Fc domain of the dual targeting antigen binding molecule according to the invention exhibits reduced binding affinity to an Fc receptor and/or reduced effector function, as compared to a native IgG Fc domain. In one such embodiment the dual targeting antigen binding molecule exhibits less than 50%, 40%, 30%, 20%, 10%, 5% of the binding affinity to an Fc receptor, and/or less than 50%, 40%, 30%, 20%, 10%, 5% of effector function, as compared to a dual targeting antigen binding molecule comprising a native IgG Fc domain. In a specific embodiment the Fc receptor is a human FcyRIIIa, FcyRI or FcyRIIa, most specifically human FcyRIIIa. In one embodiment the effector function is one or more selected from the group of CDC, ADCC, ADCP, and cytokine secretion. Substantially similar binding affinity to neonatal Fc receptor (FcRn) is desirable to be preserved.

In one embodiment the amino acid mutation that reduces the binding affinity of the Fc domain to an Fc receptor and/or effector function is an amino acid substitution, for example, those as described in PCT patent application PCT/EP20 12/055393, incorporated herein by reference in its entirety. PCT/EP20 12/055393 also describes methods of preparing such mutant Fc domains and methods for determining its properties such as Fc receptor binding or effector functions.

The dual targeting antigen binding molecule of the invention may be obtained, for example, by solid-state peptide synthesis (e.g. Merrifield solid phase synthesis) or recombinant production. For recombinant production one or more polynucleotide encoding the dual targeting antigen binding molecule (fragment) is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of a dual targeting antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989).

Therapeutic Use

The dual targeting antigen binding molecule of the present invention can be used for treatment of a tumor, especially a human tumor. In particular embodiments, the dual targeting antigen binding molecule of the present invention can induce lysis of tumor cells. In particular embodiments, the dual targeting antigen binding molecule of the present invention can inhibit the growth of tumor cells.

For the treatment of the disease, the appropriate dosage of the dual targeting antigen binding molecule of the present invention (when used alone or in combination with one or more other additional therapeutic agents) will depend on the type of disease to be treated, the route of administration, the body weight of the patient, the severity and course of the disease, preventive or therapeutic purposes, previous or concurrent therapeutic interventions, the patient's clinical history and response to the dual targeting antigen binding molecule of the present invention, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The dual targeting antigen binding molecule of the present invention is suitably administered to the patient at one time or over a series of treatments. Depending on the type and severity of the disease, about 1 mg/kg to 15 mg/kg (e.g. 0.1 mg/kg-10 mg/kg) of the dual targeting antigen binding molecule of the present invention can be an initial candidate dosage for administration to the patient, whether, for example, by one or more separate administrations, or by continuous infusion. One typical daily dosage might range from about 1 mg/kg to 100 mg/kg or more, depending on the factors mentioned above. However, other dosage regimens may be useful.

The progress of the therapy is easily monitored by conventional techniques and the determination of a therapeutically effective amount is well within the capabilities of those skilled in the art, especially in light of the detailed disclosure provided herein. Dosage amount and interval may be adjusted individually to provide plasma levels of the dual targeting antigen binding molecule of the present invention which are sufficient to maintain therapeutic effect. Usual patient dosages for administration by injection range from about 0.1 to 50 mg/kg/day, typically from about 0.5 to 1 mg/kg/day.

Pharmaceutical Compositions and Article of Manufacture

The present invention is also related to a pharmaceutical composition comprising the dual targeting antigen binding molecule of the invention and a pharmaceutically acceptable carrier. The term “pharmaceutically acceptable carrier” refers to non-toxic recipients at the dosages and concentrations employed, i.e. do not produce an adverse, allergic or other untoward reaction when administered to an animal, such as, for example, a human, as appropriate. The pharmaceutically acceptable carrier includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference.

The dual targeting antigen binding molecule of the invention may be administered in combination with one or more other agents in therapy. For instance, a dual targeting antigen binding molecule of the invention may be co-administered with at least one additional therapeutic agent with complementary activities and no adverse effect. The additional therapeutic agent comprises a drug of chemotherapy for a cancer, for example, an immunomodulatory agent and a cytostatic agent. The dual targeting antigen binding molecule of the invention and the one or more other agents in therapy may be put into different containers of an article of manufacture. In certain embodiments, the article of manufacture comprises a container and a label or package insert on or associated with the container. Suitable containers include, for example, bottles, vials, syringes, IV solution bags, etc. The label or package insert indicates the uses of the dual targeting antigen binding molecule of the invention and the one or more other agents in therapy, and the methods for treating the disease in need of the dual targeting antigen binding molecule of the invention and the one or more other agents in therapy. Additionally, the article of manufacture may further comprise one or more containers containing other materials desirable from a commercial and user standpoint, including other buffers, diluents, filters, needles, and syringes.

### EXAMPLES

### Example 1 SimBody™ and SomBody™ Design and Proof-of-Concept Study

We decided to adopt a modified IgG4 isotype for the construction of the new class of TRABs, with the S228P mutation to eliminate Fab-arm exchange and the F234A and L235A mutations to reduce interaction with high-affinity Fc receptors. Importantly, just one CD3-specific binding arm was used so that the new class of TRABs can monovalently bind to T cells only with low affinity, which will not trigger T-cell activation by CD3, unless the bispecific antibody is presented to the T cell in a multivalent fashion by a target tumor cell.

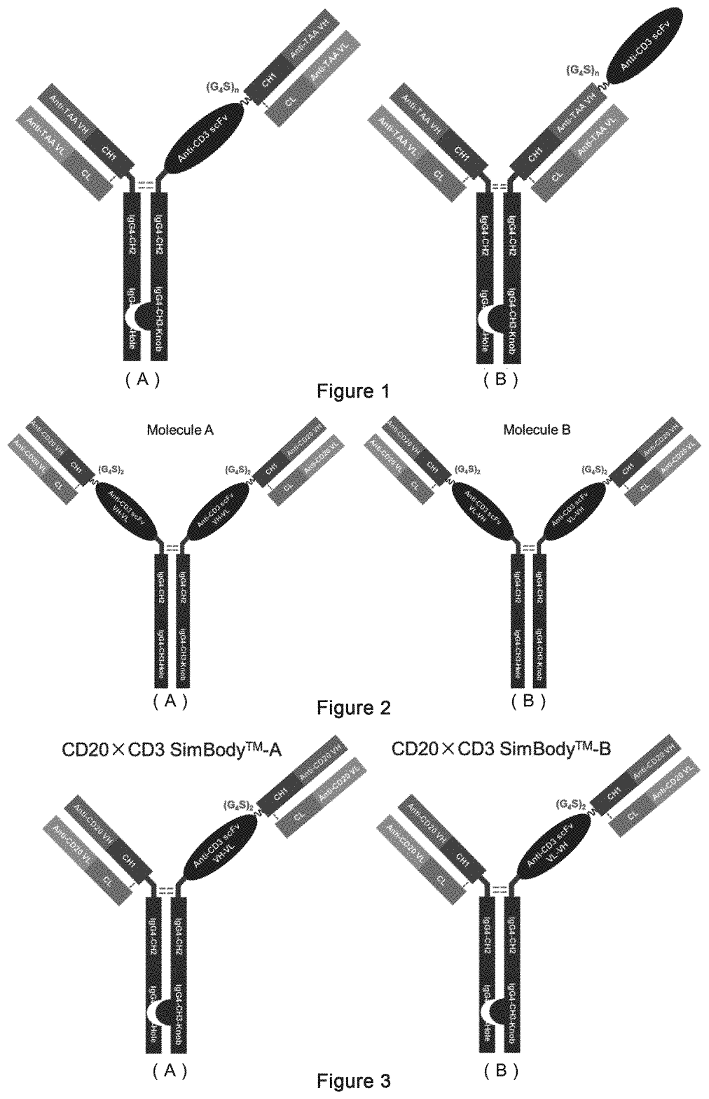

Based on these considerations, the new class of TRABs will have the following features: 1) The T cell-engaging component, to be shared by all TRABs, is an anti-CD3 scFv in either V_{H}-V_{L} or V_{L}-V_{H} orientations; 2) The tumor cell targeting component, which is different for each TRAB, is an anti-TAA IgG4 mAb with the knob (T366W) mutation in one H chain and the hole (T366S/L368A/Y407V) mutations in the other; and the anti-CD3 scFv is either 3) inserted between the anti-TAA Fab and the hinge-Fc region on the H chain of the TRAB molecule that harbors the knob mutation and is covalently connected to the C-terminus of the anti-TAA Fab (V_{H}-CH1) through a (G₄S)n-based linker, which is named SimBody™ (scFv inside of monoclonal antibody); or 4) covalently attached to the N-terminus of the anti-TAA mAb through a (G₄S)n-based linker on its H chain that harbors the knob mutation, which is named SomBody™ (scFv on top of monoclonal antibody). Co-transfection of CHO cells with cDNA encoding one L chain and two different H chains would lead to the formation of stable knob-into-hole heterodimeric IgG-like BsAb molecules (FIGS. 1A and 1B), which could be purified by protein A affinity chromatography.

As shown in FIGS. 1A and 1B, the SimBody™ and SomBody™ TRABs both contain: 1) a T cell-engaging component, an anti-CD3 scFv in either V_{H}-V_{L} or V_{L}-V_{H} orientations to be shared by all TRABs; 2) a tumor associated antigen targeting IgG4 mAb with the knob (T366W) mutation in one H chain and the hole (T366S/L368A/Y407V) mutations in the other; and (A) for SimBody™, the anti-CD3 scFv is inserted between the anti-TAA Fab and the hinge-Fc region on the H chain of the TRAB molecule that harbors the knob mutation and is covalently connected to the C-terminus of the anti-TAA Fab (V_{H}-CH1) through a (G₄S)n linker, wherein n=1 or 2; while (B) for SomBody™, the anti-CD3 scFv is covalently attached to the N-terminus of the anti-TAA mAb through a (G₄S)n-based linker on its H chain that harbors the knob mutation, wherein n=1 or 2.

A number of CD20×CD3 SimBody™ and SomBody™ TRABs (FIG. 2-5) were constructed, transiently expressed and purified, and their binding properties to both human B and T cells were analyzed by flow cytometry for proof-of-concept study. Humanized Muromonab-CD3 and FDA-approved ofatumumab sequences were used in this proof-of-concept study.

Two CD20×CD3 TRABs contained two identical heavy chains (anti-CD20-linker-anti-CD3 scFv-IgG4 fusion proteins) with anti-CD3 scFv in either V_{H}-(G₄S)₃-V_{L} or V_{L}-(G₄S)₃-V_{H} orientation and with (G₄S)n linker (n=1 or 2) between anti-CD3 scFv and anti-CD20 mAb (FIG. 2, Molecule A and Molecule B). Two CD20×CD3 SimBody™ TRABs contained two different H chains: one H chain comprised of anti-CD20 V_{H} and IgG4 CH with the S228P, F234A, L235A, T366S, L368A and Y407V mutations, and the other comprised of anti-CD20 Fab (V_{H}-CH1), (G₄S)₂, anti-CD3 scFv in either V_{H}-(G₄S)₃-V_{L} or V_{L}-(G₄S)₃-V_{H} orientation, and IgG4 (hinge-CH2-CH3) with the S228P, F234A, L235A and T366W mutations (FIG. 3, CD20×CD3 SimBody™-A and CD20×CD3 SimBody™-B).

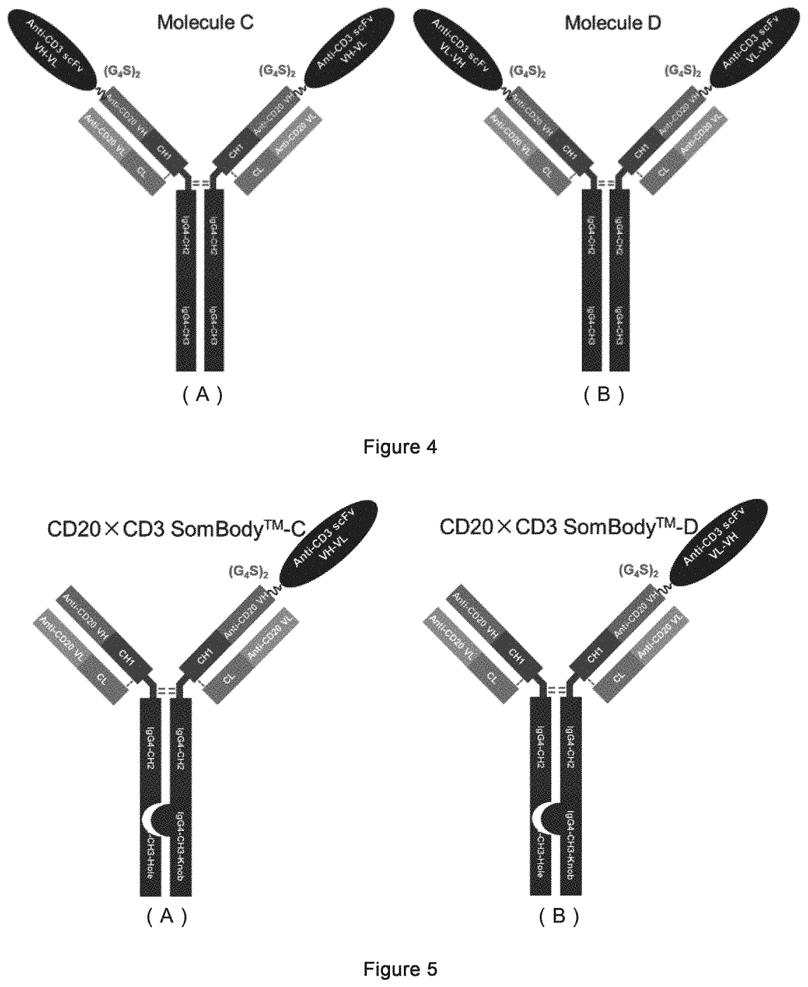

Two additional CD20×CD3 TRABs (Molecule C and Molecule D) contained two identical heavy chains (anti-CD3 scFv-(G₄S)n linker-anti-CD20-IgG4PAA fusion proteins) with anti-CD3 scFv in either V_{H}-(G₄S)₃-V_{L} or V_{L}-(G₄S)₃-V_{H} orientation and with (G₄S)n linker (n=1 or 2) between anti-CD3 scFv and anti-CD20 mAb (FIG. 4). Lastly, two CD20×CD3 SomBody™ TRABs contained two different H chains: one H chain comprised of anti-CD20 V_{H} and IgG4 CH with the S228P, F234A, L235A, T366S, L368A and Y407V mutations, and the other comprised of anti-CD20 Fab (V_{H}-CH1), (G₄S)2, anti-CD3 scFv in either V_{H}-(G₄S)₃-V_{L} or V_{L}-(G₄S)₃-V_{H} orientation, and IgG4 (hinge-CH2-CH3) with the S228P, F234A, L235A and T366W mutations (FIG. 5, CD20×CD3 SomBody™-A and CD20×CD3 SomBody™-B). The key sequences of anti-CD20 Fab and anti-CD3 scFv are listed below.

| Description of the sequence | Sequence number |
|---|---|
| The nucleotide sequence coding for V_{H} of anti-CD20 | SEQ ID NO:1 |
| Fab | |
| The amino acid sequence of V_{H} of anti-CD20 | SEQ ID NO:2 |
| Fab | |
| The amino acid sequence of CDR1 in V_{H} of anti-CD20 | SEQ ID NO:3 |
| Fab | |
| The amino acid sequence of CDR2 in V_{H} of anti-CD20 | SEQ ID NO:4 |
| Fab | |
| The amino acid sequence of CDR3 in V_{H} of anti-CD20 | SEQ ID NO:5 |
| Fab | |
| The nucleotide sequence coding for V_{L} of anti-CD20 | SEQ ID NO:6 |
| Fab | |
| The amino acid sequence of V_{L} of anti-CD20 | SEQ ID NO:7 |
| Fab | |
| The amino acid sequence of CDR1 in V_{L} of anti-CD20 | SEQ ID NO:8 |
| Fab | |
| The amino acid sequence of CDR2 in V_{L} of anti-CD20 | SEQ ID NO:9 |
| Fab | |
| The amino acid sequence of CDR3 in V_{L} of anti-CD20 | SEQ ID NO:10 |
| Fab | |
| The nucleotide sequence coding for V_{H} of anti-CD3 | SEQ ID NO:11 |
| scFV (#1) | |
| The amino acid sequence of V_{H} of anti-CD3 scFV | SEQ ID NO:12 |
| (#1) | |
| The amino acid sequence of CDR1 in V_{H} of anti-CD3 | SEQ ID NO:13 |
| scFV (#1) | |
| The amino acid sequence of CDR2 in V_{H} of anti-CD3 | SEQ ID NO:14 |
| scFV (#1) | |
| The amino acid sequence of CDR3 in V_{H} of anti-CD3 | SEQ ID NO:15 |
| scFV (#1) | |
| The nucleotide sequence coding for V_{L} of anti-CD3 | SEQ ID NO:16 |
| scFV (#1 and #2) | |
| The amino acid sequence of V_{L} of anti-CD3 scFV | SEQ ID NO:17 |
| (#1 and #2) | |
| The amino acid sequence of CDR1 in V_{L} of anti-CD3 | SEQ ID NO:18 |
| scFV (#1 and #2) | |
| The amino acid sequence of CDR2 in V_{L} of anti-CD3 | SEQ ID NO:19 |
| scFV (#1 and #2) | |
| The amino acid sequence of CDR3 in V_{L} of anti-CD3 | SEQ ID NO:20 |
| scFV (#1 and #2) | |
| The nucleotide sequence coding for V_{H} of anti-CD3 | SEQ ID NO:21 |
| scFV (#2) | |
| The amino acid sequence of V_{H} of anti-CD3 scFV | SEQ ID NO:22 |
| (#2) | |

SEQ ID NO: 1
Gaagtgcagctggtggagtctgggggaggcttggtacagcctggcaggtcc
ctgagactctcctgtgcagcctctggattcacctttaatgattatgccatg
cactgggtccggcaagctccagggaagggcctggagtgggtctcaactatt
agaggaatagtggttccataggctatgcggactctgtgaagggccgattca
ccatctccagagacaacgccaagaagtccctgtatctgcaaatgaacagtc
tgagagctgaggacacggccttgtattactgtgcaaaagatatacagtacg
gcaactactactacggtatggacgtctggggccaagggaccacggtcaccg
tctcctca
SEQ ID NO: 2
EVQLVESGGGLVQPGRSLRLSCAASGFTFNDYAMHWVRQAPGKGLEWVSTI
SWNSGSIGYADSVKGRFTISRDNAKKSLYLQMNSLRAEDTALYYCAKDIQY
GNYYYGMDVWGQGTTVTVSS
SEQ ID NO: 3
GFTFNDYA
SEQ ID NO: 4
ISWNSGSI
SEQ ID NO: 5
AKDIQYGNYYYGMDV
SEQ ID NO: 6
Gaaattgtgttgacacagtctccagccaccctgtctagtctccaggggaaa
gagccaccctctcctgcagggccagtcagagtgttagcagctacttagcct
ggtaccaacagaaacctggccaggctcccaggctcctcatctatgatgcat
ccaacagggccactggcatcccagccaggttcagtggcagtgggtctggga
cagacttcactctcaccatcagcagcctagagcctgaagattttgcagttt
attactgtcagcagcgtagcaactggccgatcaccacggccaagggacacg
actggagattaaa
SEQ ID NO: 7
EIVLTQSPATLSLSPGERATLSCRASQSVSSYLAWYQQKPGQAPRLLIYDA
SNRATGIPARFSGSGSGTDFTLTISSLEPEDFAVYYCQQRSNWPITFGQGT
RLEIK
SEQ ID NO: 8
QSVSSY
SEQ ID NO: 9
DAS
SEQ ID NO: 10
QQRSNWPIT
SEQ ID NO: 11
caggtgcagctggtgcagagcggcggcggcgtggtgcagcccggccgcagc
ctgcgcctgagctgcaaggccagcggctacaccttcacccgctacaccatg
cactgggtgcgccaggcccccggcaagggcctggagtggatcggctacatc
aaccccagccgcggctacaccaactacaaccagaaggtgaaggaccgcttc
accatcagcaccgacaagagcaagagcaccgccttcctgcagatggacagc
ctgcgccccgaggacaccgccgtgtactactgcgcccgctactacgacgac
cactactcgctggactactggggccagggcacccccgtgaccgtgtcctca
SEQ ID NO: 12
QVQLVQSGGGVVQPGRSLRLSCKASGYTFTRYTMHWVRQAPGKGLEWIGYI
NPSRGYTNYNQKVKDRFTISTDKSKSTAFLQMDSLRPEDTAVYYCARYYDD
HYSLDYWGQGTPVTVSS
SEQ ID NO: 13
GYTFTRYT
SEQ ID NO: 14
INPSRGYT
SEQ ID NO: 15
ARYYDDHYSLDY
SEQ ID NO: 16
Gacatccagatgacccagagccccagcagcctgagcgccagcgtgggcgac
cgcgtgaccatcacctgcagcgccagcagcagcgtgagctacatgaactgg
taccagcagacccccggcaaggcccccaagcgctggatctacgacaccagc
aagctggccagcggcgtgcccagccgcttcagcggcagcggcagcggcacc
gactacaccttcaccatcagcagcctgcagcccgaggacatcgccacctac
tactgccagcagtggagcagcaaccccttcaccttcggccagggcaccaag
ctgcagatcacc
SEQ ID NO: 17
DIQMTQSPSSLSASVGDRVTITCSASSSVSYMNWYQQTPGKAPKRWIYDTS
KLASGVPSRFSGSGSGTDYTFTISSLQPEDIATYYCQQWSSNPFTFGQGTK
LQIT
SEQ ID NO: 18
SSVSY
SEQ ID NO: 19
DTS
SEQ ID NO: 20
QQWSSNPFT
SEQ ID NO: 21
caggtgcagctggtgcagagcggcggcggcgtggtgcagcccggccgcagc
ctgcgcctgagctgcaaggccagcggctacaccttcacccgctacaccatg
cactgggtgcgccaggcccccggcaagggcctggagtggatcggctacatc
aaccccagccgcggctacaccaactacaaccagaaggtgaaggaccgcttc
accatcagccgcgacaatagcaagaacaccgccttcctgcagatggacagc
ctgcgccccgaggacaccggcgtgtacttctgcgcccgctactacgacgac
cactactcgctggactactggggccagggcacccccgtgaccgtgtcctca
SEQ ID NO: 22
QVQLVQSGGGVVQPGRSLRLSCKASGYTFTRYTMHWVRQAPGKGLEWIGYI
NPSRGYTNYNQKVKDRFTISRDNSKNTAFLQMDSLRPEDTGVYFCARYYDD
HYSLDYWGQGTPVTVSS

### Example 2 Plasmid Construction of the SimBody™ or SomBody™

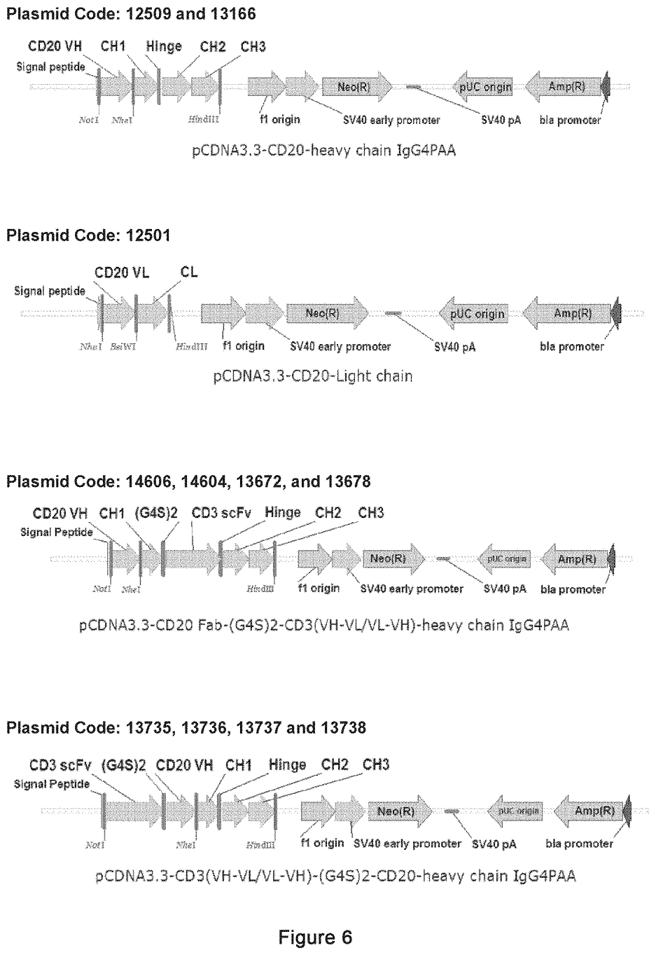

The plasmids encoding the respective heavy and light chains for making the CD20×CD3 SimBody™ or CD20×CD3 SomBody™ TRABs were constructed (as shown in FIG. 6) and listed in the below table (Table 1).

TABLE 1
Plasmids constructed to produce CD20 × CD3 SimBody ™ or
CD20 × CD3 SomBody ™ TRABs

| Plasmid code | Description |
|---|---|
| 12509 | pCDNA3.3-CD20-heavy chain IgG4PAA hole |
| 12501 | pCDNA3.3-CD20-Light chain |
| 13166 | pCDNA3.3-CD20-heavy chain IgG4PAA |
| 14606 | pCDNA3.3-CD20 Fab-(G₄S)₂-CD3(V_{H}-V_{L})- |
| | heavy chain IgG4PAA |
| 13672 | pCDNA3.3-CD20 Fab-(G₄S)₂-CD3(V_{L}-V_{H})- |
| | heavy chain IgG4PAA |
| 14604 | pCDNA3.3-CD20 Fab-(G₄S)₂-CD3(V_{H}-V_{L})- |
| | heavy chain IgG4PAA knob |
| 13678 | pCDNA3.3-CD20 Fab-(G₄S)₂-CD3(V_{L}-V_{H})- |
| | heavy chain IgG4PAA knob |
| 13735 | pCDNA3.3-CD3(V_{H}-V_{L})-(G₄S)₂-CD20-heavy |
| | chain IgG4PAA |
| 13736 | pCDNA3.3-CD3(V_{L}-V_{H})-(G₄S)₂-CD20-heavy |
| | chain IgG4PAA |
| 13737 | pCDNA3.3-CD3(V_{H}-V_{L})-(G₄S)₂-CD20-heavy |
| | chain IgG4PAA knob |
| 13738 | pCDNA3.3-CD3(V_{L}-V_{H})-(G₄S)₂-CD20-heavy |
| | chain IgG4PAA knob |

Briefly, the plasmids containing the anti-CD20 heavy chain-IgG4PAA (S228P, F234A, L235A) and hole (T366S, L368A, Y407V) mutations (#12509) & light chain (#12501) were synthesized and constructed into pCDNA3.3 plasmid using the restriction enzyme Not I/Hind III or NheI I/Hind III sites. Plasmid #13166 containing the anti-CD20 heavy chain-IgG4PAA was obtained through point mutations of the following amino acids (S366T, A368L, V407Y) from plasmid #12509 using Q5® Site-Directed Mutagenesis Kit (New England Biolabs, Catlog #E0552S) using the following primers respectively:

Forward primer:
SEQ ID NO: 22
5′-GGTCAGCCTGACCTGCCTGGTCAAAGGCT-3′;
Reverse primer:
SEQ ID NO: 22
5′-TGGTTCTTGGTCATCTCCTCCTGGGATG-3′;
Forward primer:
SEQ ID NO: 22
5′-CTTCTTCCTCTACAGCAGGCTAACCG-3′;
Reverse primer:
SEQ ID NO: 22
5′-GAGCCGTCGGAGTCCAGCACGGGAGGC-3′).

The DNA sequences of CH1-(G₄S)₂-anti-CD3 scFv (V_{H}-V_{L} or V_{L}-V_{H})-hinge-CH2-CH3 IgG4 (containing 5228P, F234A, L235A mutations) fragments were synthesized and cloned into plasmid #12509 via the NheI and HindIII restriction enzyme sites to replace the original CH1-hinge-CH2-CH3 region to generate plasmids #14606 and #13672. The DNA sequences of anti-CD3 (#1 or #2) scFv (V_{H}-V_{L} or V_{L}-V_{H})-(G₄S)2-anti-CD20 V_{H} fragments were synthesized and cloned into plasmid #13166 using the restriction enzyme Not I and NheI sites to generate plasmids #13735 and #13736.

The plasmids #14604, #13678, #13737 and #13738 harboring the knob mutation (T366W) were generated via Q5® Site-Directed Mutagenesis Kit (New England Biolabs, Catlog #E0552S) using the following primers respectively:

(1) Forward primer:
SEQ ID NO: 22
5′-AACCAGGTCAGCCTGTGGTGCCTGGTCAAAGGCTTCTACC-3′, ,
and
(2) Reverse primer:
SEQ ID NO: 22
5′-CTTGGTCATCTCCTCCTGGGATGGGGGCAGGGTGTACA-3′,

to introduce the T366W mutation to the plasmids #14606, #13678, #13735 and #13736.

### Example 3 Expression, Purification and SDS-PAGE Analysis

The plasmids encoding the heavy and light chains were mixed as listed in the table 2 and co-transfected into 200 ml Expi-CHO-S cells (Thermo Fisher, cat #A29127)/each combination. The transfected Expi-CHO-S cells were allowed for culture at 37° C. containing 5% CO₂ incubator for 8-10 days before harvest. The antibodies were purified from cell culture supernatant using the AKTA systems (GE Healthcare) via MabSelect Sure Protein A agarose column (GE Healthcare, cat #GE-17543804) according to the manufacturer's protocol. The antibodies were eluted, and buffer exchanged to 1×PBS. For SimBody™ and SomBody™ TRABs, a further purification step using the GE HiTrap HP-SP cation exchange column (GE Healthcare, cat #29051324) were applied according to manufacturer's protocol. Different elution fractions were collected and evaluated by SDS-PAGE, size excusive chromatography (SEC-HPLC), and non-reduced capillary electrophoresis (NR-CE-SDS) analyses to facilitate the optimization of the elution conditions for SimBody™ and SomBody™ TRABs. The total antibody yields were calculated based upon respective extinction coefficient at 280 nm using a NanoPhotometer instrument (Implen, NanoPhotometer® NP80-Touch).

TABLE 2
Plasmids combinations for co-transfection in Expi-CHO-S cells

| Test Articles | Plasmids | Descriptions |
|---|---|---|
| Molecule A | 14606 | pCDNA3.3-CD20 Fab-(G₄S)₂-CD3(V_{H}-V_{L})- |
| | | heavy chain IgG4PAA |
| | 12501 | pCDNA3.3-CD20-Light chain |
| Molecule B | 13672 | pCDNA3.3-CD20 Fab-(G₄S)₂-CD3(V_{L}-V_{H})- |
| | | heavy chain IgG4PAA |
| | 12501 | pCDNA3.3-CD20-Light chain |
| CD20 × CD3 | 14604 | pCDNA3.3-CD20 Fab-(G₄S)₂-CD3(V_{H}-V_{L})- |
| SimBody ™- | | heavy chain IgG4PAA knob |
| A | 12509 | pCDNA3.3-CD20-heavy chain IgG4PAA |
| | | hole |
| | 12501 | pCDNA3.3-CD20-Light chain |
| CD20 × CD3 | 13678 | pCDNA3.3-CD20 Fab-(G₄S)₂-CD3(V_{L}-V_{H})- |
| SimBody ™- | | heavy chain IgG4PAA knob |
| B | 12509 | pCDNA3.3-CD20-heavy chain IgG4PAA |
| | | hole |
| | 12501 | pCDNA3.3-CD20-Light chain |
| Molecule C | 13735 | pCDNA3.3-CD3(V_{H}-V_{L})-(G₄S)₂-CD20- |
| | | heavy chain IgG4PAA |
| | 12501 | pCDNA3.3-CD20-Light chain |
| Molecule D | 13736 | pCDNA3.3-CD3(V_{L}-V_{H})-(G₄S)₂-CD20- |
| | | heavy chain IgG4PAA |
| | 12501 | pCDNA3.3-CD20-Light chain |
| CD20 × CD3 | 13737 | pCDNA3.3-CD3(V_{H}-V_{L})-(G₄S)₂-CD20- |
| SomBody ™- | | heavy chain IgG4PAA knob |
| C | 12509 | pCDNA3.3-CD20-heavy chain IgG4PAA |
| | | hole |
| | 12501 | pCDNA3.3-CD20-Light chain |
| CD20 × CD3 | 13738 | pCDNA3.3-CD3(V_{L}-V_{H})-(G₄S)₂-CD20- |
| SomBody ™- | | heavy chain IgG4PAA knob |
| D | 12509 | pCDNA3.3-CD20-heavy chain IgG4PAA |
| | | hole |
| | 12501 | pCDNA3.3-CD20-Light chain |

3 μg/each of the test articles were prepared in 4× sample buffer (Life Technology, Catlog #NP007) and boiled at 65° C. before loaded onto SDS-PAGE gel to evaluate the protein purity. 8% non-reducing SDS-PAGE analysis was used to evaluate the test articles in their native condition and 10% iodoacetamide (JAM) were added to samples.

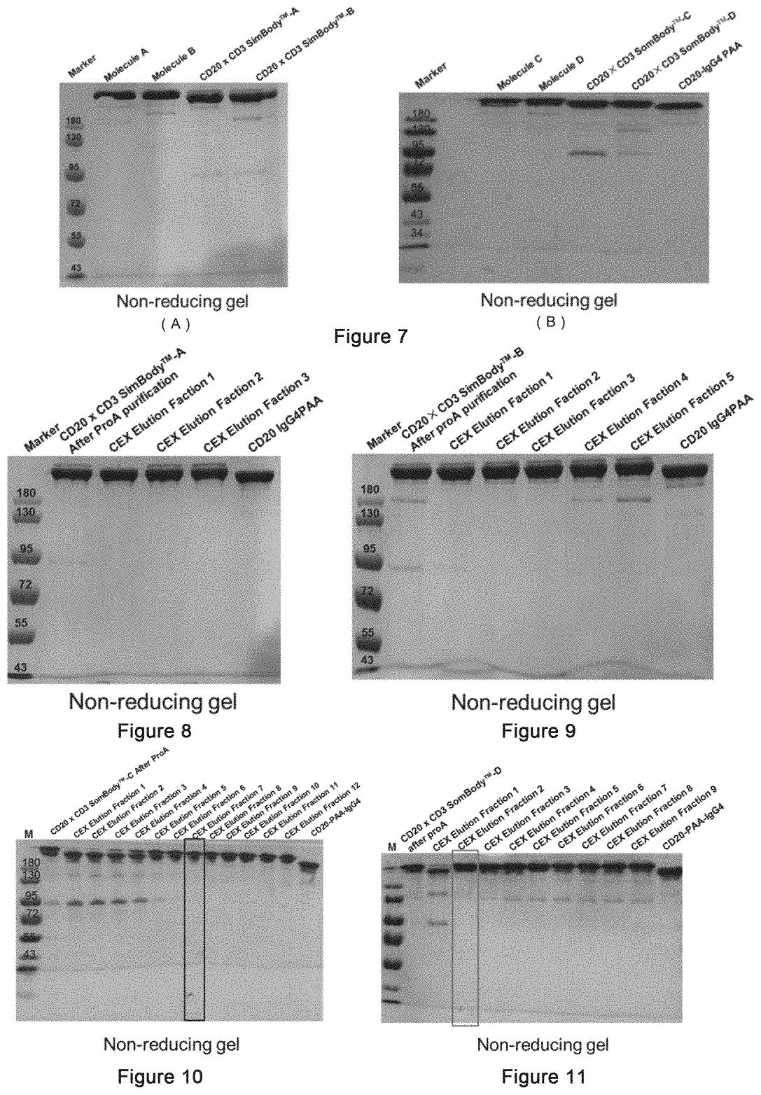

The purities of the test articles in their native conditions after one-step protein A column purification were shown as in FIG. 7. All test articles appeared as one major band sizes above 180KDa marker. There were lower molecular weight bands between 95 to >180 KDa for CD20×CD3 SimBody™ and SomBody™ TRABs.

CD20×CD3 SimBody™ and SomBody™ were further subjected to cation exchange purification and different elution fractions were collected and evaluated on non-reducing SDS-PAGE (FIG. 8-11). The various elution fractions from cation exchange purification for CD20×CD3 SimBody™-A (FIG. 8), CD20×CD3 SimBody™-B (FIG. 9), CD20×CD3 SomBody™-C (FIG. 10) and CD20×CD3 SomBody™-D (FIG. 11) were analyzed under non-reducing conditions on 8% SDS-poly acrylamide gel. CEX elution fraction 1 from SimBody™-A, CEX elution fraction 3 from SimBody™-B, CEX elution fraction 7 from SomBody™-C and CEX elution fraction 2 from SomBody™-D were selected for further quality analyses.

### Example 4 Purity Analysis by SEC-HPLC

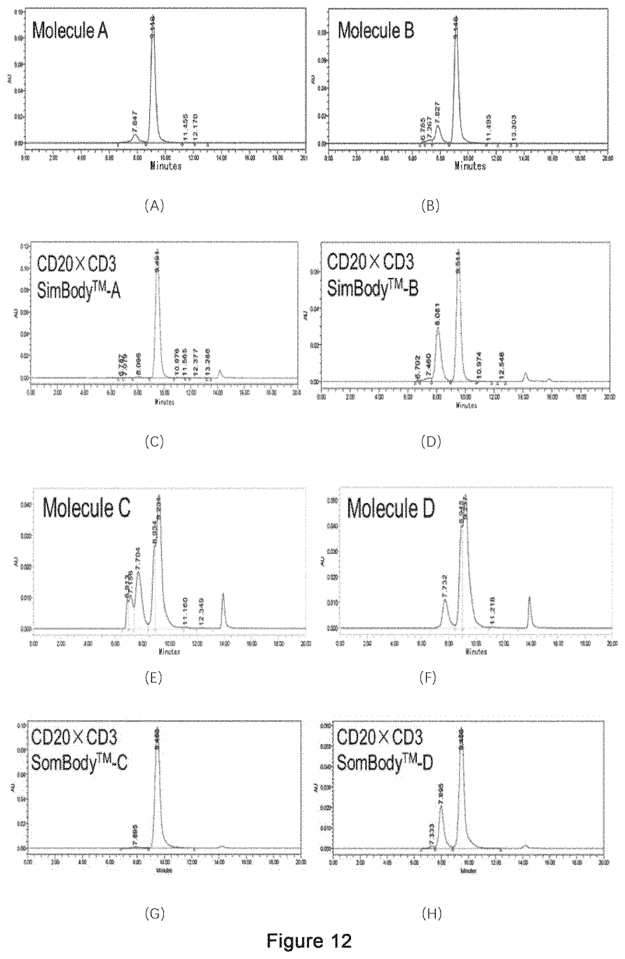

Size exclusion chromatography (SEC) coupled with HPLC was used to profile the qualities of the test articles under their native conditions. The test articles were diluted in ddH₂O to a concentration of 1 mg/mL. SEC experiments were performed using Agilent 1200 and TSKgel G3000SWXL. The mobile phase was 50 mM PB solution (pH 7.0) and 300 mM NaCl at a flow rate of 0.8 ml/min. The UV absorbance was measured at a wavelength of 280 nm. Data analyses were performed using Waters Empower 3 software. As shown in FIG. 12, CD20×CD3 SimBody™-A and SomBody™-C resolved a clear main peak over 98% with little LMW fragments or HMW aggregates after cation exchange purification. However, CD20×CD3 SimBody™-B and SomBody™-D contained a large fraction of HMW aggregates (>25%) even after cation exchange purification. The percentages of HMW aggregates, monomer main peak and LMW fragments for all test articles were summarized in Table 3.

TABLE 3
Results of CD20 × CD3 SimBody ™ and
SomBody ™ SEC-HPLC

| Test Articles | HMW % | Main Peak % | LMW % |
|---|---|---|---|
| Molecule A | 9.6 | 90.06 | 0.33 |
| Molecule B | 18.06 | 81.84 | 0.10 |
| CD20 X CD3 SimBody ™-A | 1.33 | 98.18 | 0.49 |
| CD20 X CD3 SimBody ™-B | 35.39 | 64.40 | 0.21 |
| Molecule C | 51.5 | 47.83 | 0.67 |
| Molecule D | 38.49 | 60.84 | 0.67 |
| CD20 X CD3 SomBody ™-C | 1.91 | 98.09 | — |
| CD20 X CD3 SomBody ™-D | 26.84 | 73.16 | — |

### Example 5 Purity Analysis by NR- and R-CE SDS

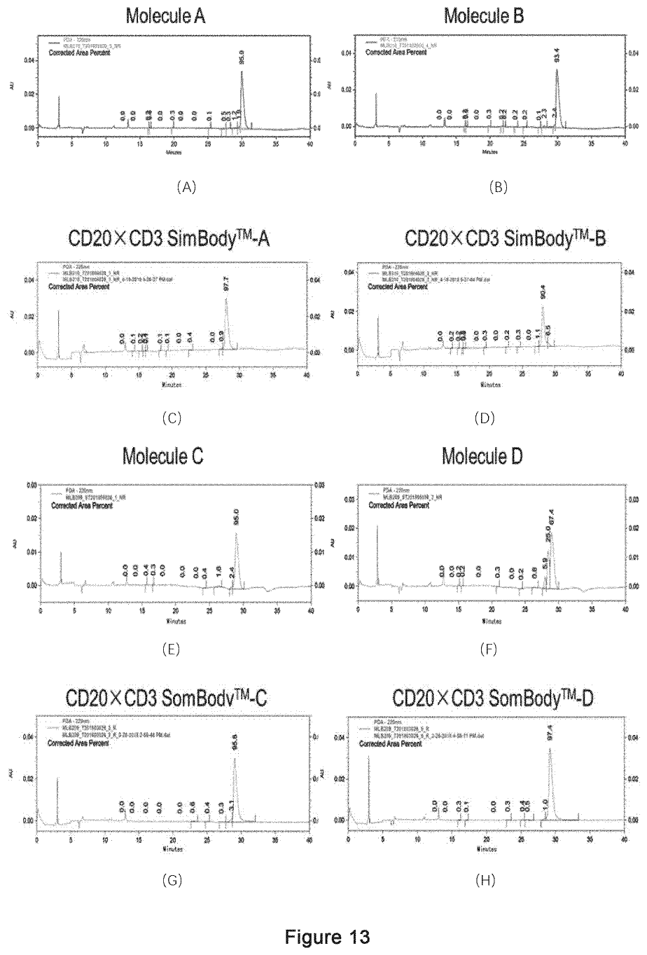

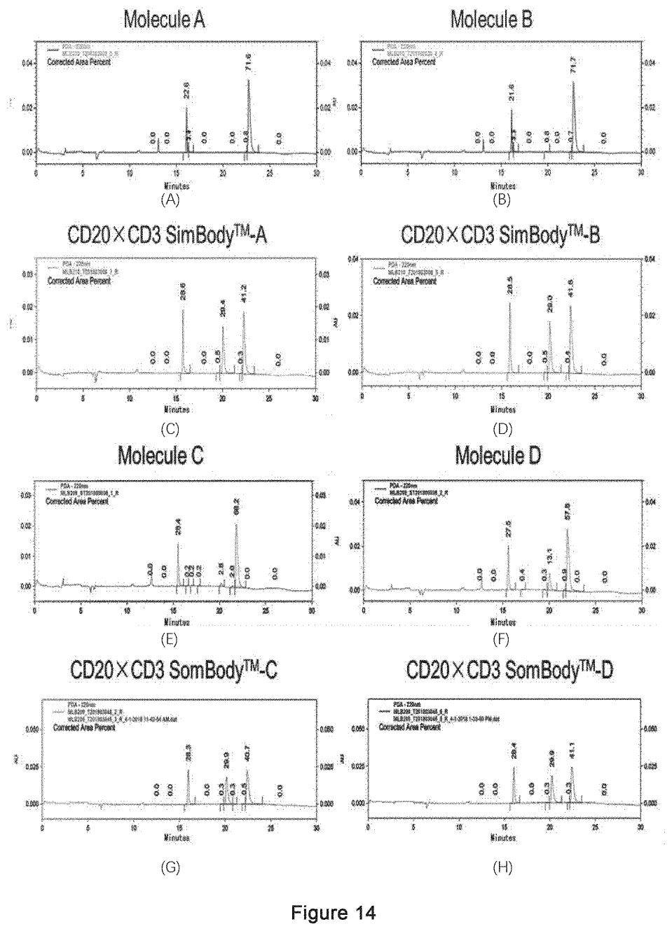

CE-SDS analysis was used to characterize the purity of the test articles. Briefly, the Beckman PA800 plus system was used for CE-SDS with Beckman capillary (50 um ID×20 cm) to analyze the test articles. For non-reducing CE-SDS analysis, all test articles were diluted to 1.0 mg/mL in 1% SDS-Tris-HCl solution containing 5% iodoacetamide (0.245M). For reducing CE-SDS analysis, all test articles were diluted to 1.0 mg/mL in SDS and (3-ME containing 0.1 M tris-HCl buffer (pH 9.0). A 10 KDa internal standard was added to all test article mixtures and heated at 70° C. for 5 min before injection. The instrument was operated at a voltage of 15 kV for protein separation and the UV detection was recorded at a wavelength of 220 nm. The results indicated that under NR-CE-SDS conditions, Molecule A, Molecule B, Molecule C and all the SimBody™ and SomBody™ TRABs displayed high purity over 90%; Molecule D contained large proportions of LMW fragments after similar purification process. Under the R-CE-SDS conditions, all test articles displayed similar results as the SDS-PAGE. The NR- and R-CE-SDS results were summarized in FIGS. 13-14 and table 4-5.

Results from non-reducing (NR) CE-SDS (FIG. 13) analysis showed Molecule A contained 95% monomer and 5.0% fragments; Molecule B showed a slightly lower percentage of monomer (93.4%) and more fragments (6.6%). Both molecules showed high percentages of main peak in the R-CE-SDS analysis (see summary results in Table 4). Both CD20×CD3 SimBody™-A and -B showed three major peaks in N-CE-SDS indicating light chain, and two heavy chains of different molecular sizes respectively (FIG. 14), the percentages of each peak were summarized in Table 5.

TABLE 4
Result of purity analysis of SimBody ™ and
SomBody ™ by NR-CE -SDS

| | NR-CE-SDS | |
|---|---|---|
| Test Articles | Main Peak | Others |
| Molecule A | 95.0% | 5.0% |
| Molecule B | 93.4% | 6.6% |
| CD20 X CD3 SimBody ™-A | 97.7% | 2.3% |
| CD20 X CD3 SimBody ™-B | 90.4% | 9.6% |
| Molecule C | 95.0% | 5.0% |
| Molecule D | 67.4% | 33.6% |
| CD20 X CD3 SomBody ™-C | 95.6% | 4.4% |
| CD20 X CD3 SomBody ™-D | 97.4% | 2.6% |

TABLE 5
Result of purity analysis of SimBody ™ and
SomBody ™ by R-CE-SDS

| | R-CE-SDS | | | |
|---|---|---|---|---|
| Test Articles | Peak 1 | Peak 2 | Peak 3 | Others |
| Molecule A | 22.6% | 71.6% | / | 5.8% |
| Molecule B | 21.6% | 71.7% | / | 6.7% |
| CD20 X CD3 SimBody ™-A | 28.6% | 29.4% | 41.2% | 0.8% |
| CD20 X CD3 SimBody ™-B | 28.5% | 29.0% | 41.6% | 0.9% |
| Molecule C | 28.4% | 66.2% | / | 5.4% |
| Molecule D | 27.5% | 57.8% | 13.1% | 1.6% |
| CD20 X CD3 SomBody ™-C | 28.3% | 29.9% | 40.7% | 1.1% |
| CD20 X CD3 SomBody ™-D | 28.4% | 29.9% | 41.1% | 0.6% |

### Example 6 Assessment of Binding Properties to Both Human B and T Cells

The human B cells (Raji) or T cells (Jurkat) were washed once and re-suspended in 1×PBS+1% FBS and then seeded at 1×10⁵/well, 50 μl/well in the 96-well-plate. The cells were incubated with 50 μl/well serial diluted test articles (3-fold serially diluted from 10 ug/mL) on ice for 60 min and then centrifuged, washed 2 times by ice-cold 1×PBS+1% FBS. The cells were then incubated with 100 μl/well detection antibody anti-hIgG-PE (1:200 diluted) on ice for 60 min, centrifuged and washed 2 times by ice-cold 1×PBS+1% FBS and re-suspended with 200 μL PBS+1% FBS. The binding properties to human B and T cells under various concentrations of test articles were accessed by MFI (mean fluorescent intensity) signals measured by flow cytometry analysis. Live cells were gated based on light scatter properties in the flow cytometer and CD3-positive Jurkat cell or CD20-positive Raji cell populations in the gated live cells were identified by negative staining (the action of PBS instead of test articles). Each concentration of the test articles was tested in duplicates, and the MFI∓stand error mean (SEM) was used to represent that concentration and plotted against the logarithmic concentrations of the test articles to generate a non-linear regression curve-fit using the Graphpad® Prism 6 software. The half maximal effective concentration (EC50) of the test articles to bind human B or T cells was calculated from the respective dose-response curves.

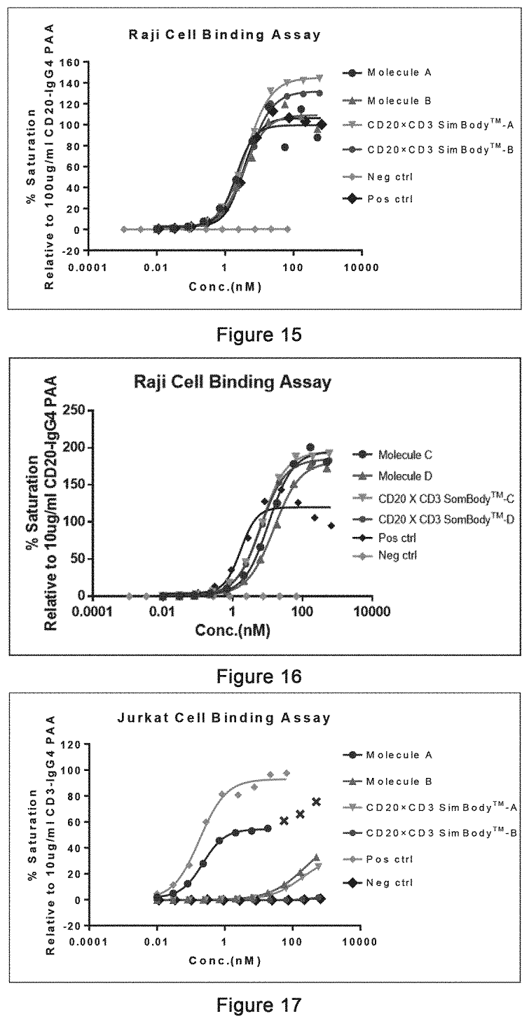

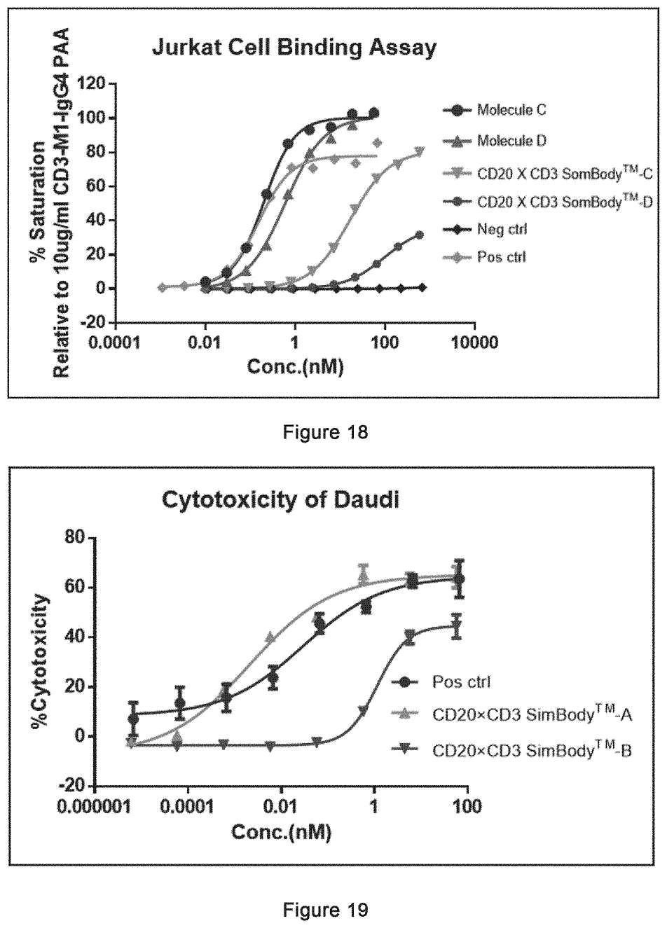

All the test articles (CD20×CD3 SimBody™ and SomBody™) induced the florescence signals in the human B cells (Raji) in a concentration-dependent manner and showed comparable binding activities as suggested by their respective EC50 (FIGS. 15, 16 & Table 6, 7). All CD20×CD3 SimBody™ and SomBody™ showed higher binding plateau than the positive control (anti-CD20 IgG4PAA).

TABLE 6
Binding activities of CD20 × CD3 SimBodies to
CD20⁺ Raji cells

| Test/Control Articles | EC50(nM) | Lot |
|---|---|---|
| Molecule A | 2.11 | 20180225 |
| Molecule B | 3.45 | 20180225 |
| CD20 × CD3 SimBody ™-A | 4.09 | 20180301 |
| CD20 × CD3 SimBody ™-B | 4.70 | 20180302 |
| Neg ctrl (CD3-M1-IgG4 PAA) | >67 | 20171010 |
| Pos ctrl (CD20-IgG4 PAA) | 3.26 | 20171113 |

TABLE 7
The EC50 of the binding activities of CD20 × CD3
SomBodies to CD20-positive Raji cells

| Test/Control Articles | EC50(nM) | Lot |
|---|---|---|
| Molecule C | 10.76 | 20180319 |
| Molecule D | 15.23 | 20180319 |
| CD20 × CD3 SomBody ™-C | 6.672 | 20180321 |
| CD20 × CD3 SomBody ™-D | 6.425 | 20180322 |
| Neg ctrl (CD3-M1-IgG4 PAA) | >67 | 20171010 |
| Pos ctrl (CD20-IgG4 PAA) | 1.694 | 20171113 |

Comparing to the positive control (anti-CD3 M1 IgG4PAA), all the CD20×CD3 SimBody™ and SomBody™ displayed decreased binding activities to human T cells (Jurkat) at different degrees (FIGS. 17 & 18). The binding EC50 to Jurkat cells were summarized in Table 8.

TABLE 8
The EC50 of the binding activities of CD20 × CD3
SimBodies to CD3-positive Jurkat cells

| Test/Control Articles | EC50(nM) | Lot |
|---|---|---|
| Molecule A | 0.22 | 20180225 |
| Molecule B | >503 | 20180225 |
| CD20 × CD3 SimBody ™-A | >581 | 20180301 |
| CD20 × CD3 SimBody ™-B | >581 | 20180302 |
| Pos ctrl (CD3-M1-IgG4 PAA) | 0.18 | 20171010 |
| Neg ctrl (CD20-IgG4 PAA) | >670 | 20171113 |

TABLE 9
The EC50 of the binding activities of CD20 × CD3
SomBodies to CD3-positive Jurkat cells

| Test/Control Articles | EC50(nM) | Lot |
|---|---|---|
| Molecule C | 0.2014 | 20180319 |
| Molecule D | 0.6003 | 20180319 |
| CD20 × CD3 SomBody ™-C | 16.65 | 20180321 |
| CD20 × CD3 SomBody ™-D | 96.79 | 20180322 |
| Pos ctrl (CD3-M1-IgG4 PAA) | 0.155 | 20171010 |
| Neg ctrl (CD20-IgG4 PAA) | >670 | 20171113 |

### Example 7 Lysis of B Lymphoma Cells by Redirecting T Cells

CD20⁺ Daudi cells were maintained in RPMI 1640 culture media (Gibco, Catlog #22400-089) containing HEPES/L-Glutamine/10% FBS and used as target cells. Daudi cells were washed once with culture media and seeded in the 96-well plate at 2E4/well, 50 μl/well respectively. Human PBMCs were used as effector cells and washed once in RPMI 1640 culture media and seeded in the 96-well plate containing the target cells at 2E5 cells/well, 50 μl/well. The effector cell to target cell ratio was 10:1 (E:T=10:1). Then, 20 μl test articles (10-fold serially-diluted from 100 ug/mL) were added to the cell mixture and incubated in 37° C., 5% CO₂ for 1 day. On day 2, the assay plates were removed from 37° C. incubator and equilibrate to 22° C. before centrifugation at 350×g. 15 μl of Lysis Solution were added to the positive control wells (which contained only target cells) 30 min before the centrifugation.

50 μl supernatant aliquots were then transferred to a new 96-well clear flat bottom plate (Costar, catlog #3599) and 50 μl of the CytoTox 96® Reagent (Promega, catlog #G1780) were added to each sample aliquot. The assay plates were covered with foil or an opaque box to protect from light and incubated for 30 minutes at room temperature. The absorbance at 490 nm or 492 nm were recorded using SpectraMax. Each concentration of the test articles was tested in duplicates, and the percentages of cytotoxicity over the total target cell lysis (readout from positive controls)∓stand error mean (SEM) was used to represent that concentration and plotted against the logarithmic concentrations of the test articles to generate a non-linear regression curve-fit using the Graphpad® Prism 6 software. The half maximal effective concentration (EC50) of the test articles to redirect T cells to lyse B cells was calculated from the respective dose-response curves.

The results were summarized in FIG. 19 and Table 10.

TABLE 10
Result of lysis of Daudi by CD20 × CD3
SimBody ™ redirected T cells

| Test/Control Articles | Lot | EC50 (pM) |
|---|---|---|
| Pos ctrl (CD20 × CD3 CrossMab) | 2017 | 30 |
| CD20 × CD3 SimBody ™-A | 20180301 | 2 |
| CD20 × CD3 SimBody ™-B | 20180302 | 1150 |

### Example 8 T Cell Activation Assay

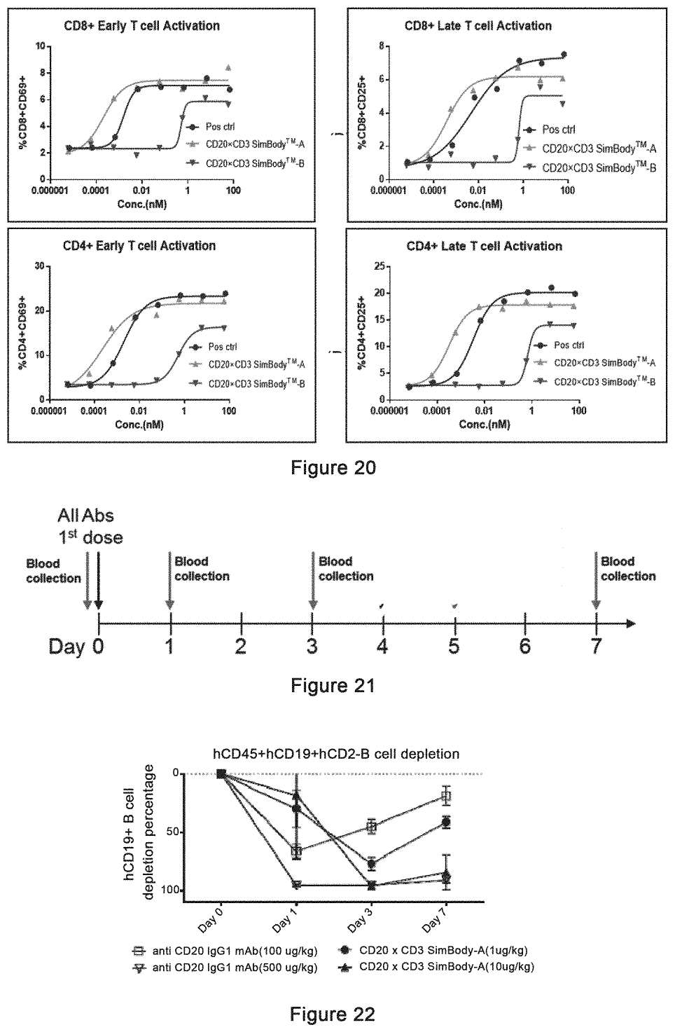

T cell activation induced by CD20×CD3 SimBody™ was evaluated together with the in vitro redirect lysis of human B lymphoma cells by human peripheral T cells assay as described in Example 7. After 50 μl supernatant were taken from each well for cytotoxicity evaluation, the remaining cells in the plates were washed once with PBS solution and stained by CD69-PE, CD25-PE, CD8-FITC, and CD4-PerCP at a concentration of 50 μl/well (1:18 dilution) in PBS+1% FBS on ice for 30 min; the cells were centrifuged and washed by PBS+1% FBS twice and resuspend with 200 μL PBS+1% FBS before evaluation on FACS machine (Guava, Millipore). The results were shown in FIG. 20 and Table 11-12.

TABLE 11
Result of CD20 × CD3 SimBody ™
induced early T cell activation

| | %CD8 + | %CD4 + |
|---|---|---|
| | CD69 + | CD69 + |
| Test/Control Articles | EC50 (pM) | EC50 (pM) |
| Pos ctrl (CD20 × CD3 1:1 CrossMab) | 1.54 | 2.02 |
| CD20 × CD3 SimBody ™-A | 0.21 | 0.23 |
| CD20 × CD3 SimBody ™-B | 474 | 523 |

TABLE 12
Result of CD20 × CD3 SimBody ™ induced
late T cell activation

| | %CD8 + | %CD4 + |
|---|---|---|
| | CD25 + | CD25 + |
| Test/Control Articles | EC50 (pM) | EC50 (pM) |
| Pos ctrl (CD20 × CD3 1:1 CrossMab) | 4.74 | 3.32 |
| CD20 × CD3 SimBody ™-A | 0.37 | 0.31 |
| CD20 × CD3 SimBody ™-B | 648.50 | 658.50 |

### Example 9 Study on the B Cell Depletion Effect In Vivo

The in vivo potency of B cell depletion by CD20×CD3 SimBody™-A was evaluated in NSG mice reconstituted by human CD34+ hematopoietic stem cells (HSC). 12 female NSG (NOD scid gamma) mice, reconstituted by hCD34+ human hematopoietic stem cells (HSCs) for 20-24 weeks to ensure stable establishment of human B/T cells. Among the mice, the average percentage of B cells is 45.89%; the average percentage of CD4+ or CD8+ T cells is 38.20% or 8.67% respectively before the start of dosing. HSC-NSG mice were intravenously dosed a single bolus of CD20×CD3 SimBody™ at 1 μg/kg or 10 μg/kg of body weight respectively, and pre- and post-dose blood samples were collected and analyzed by flow cytometry for the presence of peripheral B lymphocytes. Positive control groups using anti-CD20 IgG1 monoclonal antibody (a single bolus dose at 100 μg/kg or 500 μg/kg of body weight) were also included in the study to compare the efficacy.

Antibodies used for flow cytometry (FCM) analysis are as follows: anti-hCD19 (BioLegend, CatLog #302206); anti-hCD45 (BioLegend, CatLog #304038); anti-hCD4 (BioLegend, CatLog #300518); anti-hCD8 (BioLegend, CatLog #301032); anti-hCD2 (Biolegend, CatLog #300312).

Animals were divided into three groups and subjected to i.v. dosing according to Table 13.

TABLE 13
Preparation of test and control articles and their dosing regimen

| | | Dosing | Dosing |
|---|---|---|---|
| Group | | Level | Volume/ |
| (n = 3) | Test/Control Articles | (μg/kg) | Route |
| A | CD20 × CD3 SimBody ™-A | 1 | 100 μL/i.v. |
| B | CD20 × CD3 SimBody ™-A | 10 | 100 μL/i.v. |
| C | Anti-CD20 IgG1 monoclonal | 100 | 100 μL/i.v. |
| | antibody | | |
| D | Anti-CD20 IgG1 monoclonal | 500 | 100 μL/i.v. |
| | antibody | | |

Blood were collected for FCM analysis on day 0 before dosing and on the following days after dosing: day 1, day 3, and day 7. Briefly, 80 μL blood were collected from eye and transferred to tubes containing sodium heparin to prevent coagulation. Freshly prepared 1:1 BD Pharm Lyse: ddH₂O were used to lyse the red blood cells and the remaining cells were washed in 1000 μL FACS buffer (1×PBS, 2% FBS) twice before primary antibody incubation on ice for 30 min After two additional washes by FACS buffer, FCM analysis were performed by NovoCyte 3130 machine.

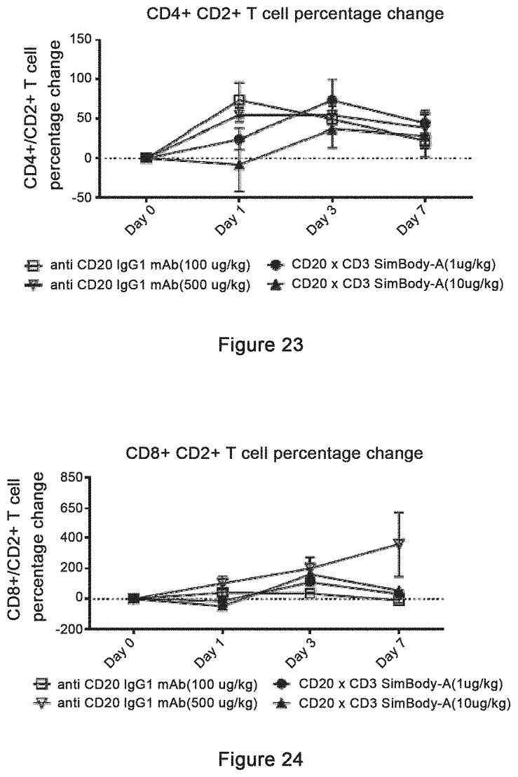

The total population of hCD45+ cells in the 80 μL blood collected per animal under various time points were gated to analyze the relative percentages of hCD19+/hCD2-B cells (FIG. 22), and hCD4+/hCD8+/hCD2+ T cells (FIG. 23 and FIG. 24). CD20×CD3 SimBody-A can effectively deplete B cells at very low doses such as 10 ug/kg for 7 days.

### Example 10 Structure Confirmation by Mass Spectrometry

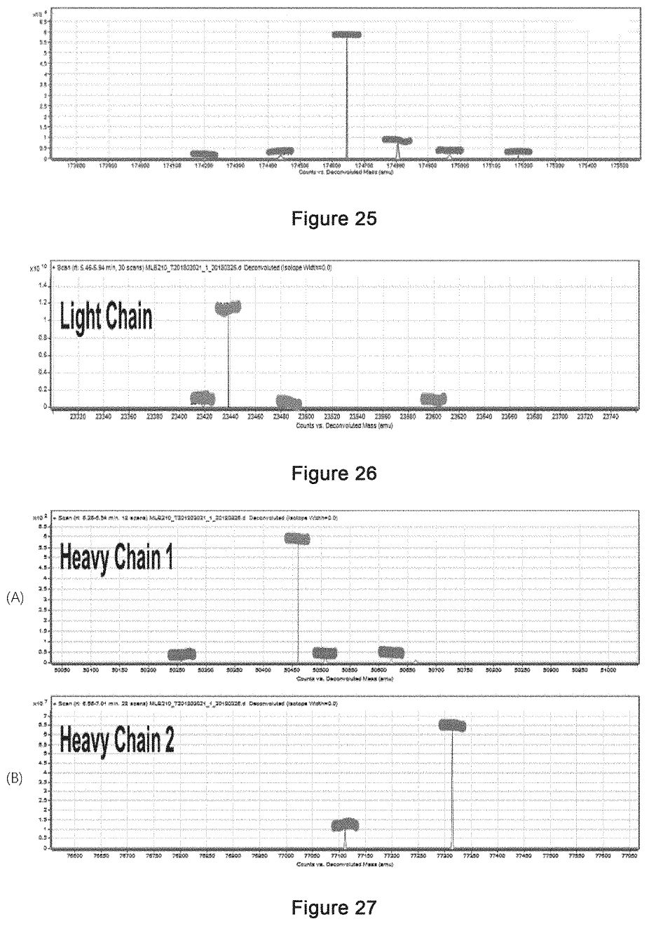

Intact mass measurements were performed to confirm the molecular weight of reduced or non-reduced CD20×CD3 SimBody™-A. Agilent 6530 Q-TOF was used for LC/MS. The test article was diluted to a concentration of 1 mg/mL in 50 μl 0.05 M tris-HCl buffer (pH 8.0). The mobile phase solution is 0.1% formic acid and 0.1% formic acid in ACN. A total of 10 μg of test article was injected for each LC/MS run. Data analysis was performed in Agilent Qualitative Analysis Bio confirm. As shown in FIG. 25-27, the difference between the total observed and theoretical molecular weight is 1.43 Da under non-reduced condition, 0.15 Da for light chain, 0.53 Da for heavy chain 1 and 0.39 Da for heavy chain 2.

1. A dual targeting antigen binding molecule, comprising a first antigen binding moiety capable of specific binding to a T cell-activating antigen, a second antigen binding moiety capable of specific binding to a target cell antigen, and an Fc domain consisting of a first and a second subunit capable of stable association,
wherein the first antigen binding moiety comprises a scFv and the second antigen binding moiety comprises a first Fab and a second Fab, wherein the scFv comprises a variable region of heavy chain (V_{H}) and a variable region of light chain (V_{L}) from the N-terminus to C-terminus of the scFv, or a variable region of light chain (V_{L}) and a variable region of heavy chain (V_{H}) from the N-terminus to C-terminus of the scFv, and wherein the second antigen binding moiety comprises a first Fab fused at the C-terminus of the Fab heavy chain to the scFv, and the second antigen binding moiety comprises a second Fab fused at the C-terminus of the Fab heavy chain to the Fc domain.

2.-4. (canceled)

5. The dual targeting antigen binding molecule of claim 1, wherein the first Fab fused at the C-terminus of the Fab heavy chain to the N-terminus of the variable region of heavy chain (V_{H}) of the scFv.

6. The dual targeting antigen binding molecule of claim 1, wherein the first Fab fused at the C-terminus of the Fab heavy chain to the N-terminus of the variable region of light chain (V_{L}) of the scFv.

7. A dual targeting antigen binding molecule, comprising a first antigen binding moiety capable of specific binding to a T cell-activating antigen, a second antigen binding moiety capable of specific binding to a target cell antigen, and an Fc domain consisting of a first and a second subunit capable of stable association, wherein the first antigen binding moiety comprises a scFv and the second antigen binding moiety comprises a first Fab and a second Fab, wherein the scFv comprises a variable region of heavy chain (V_{H}) and a variable region of light chain (V_{L}) from the N-terminus to C-terminus of the scFv, or a variable region of light chain (V_{L}) and a variable region of heavy chain (V_{H}) from the N-terminus to C-terminus of the scFv, wherein the second antigen binding moiety comprises a first Fab fused at the N-terminus of the Fab heavy chain to the scFv; and the second antigen binding moiety comprises a second Fab fused at the C-terminus of the Fab heavy chain to the Fc domain.

8. The dual targeting antigen binding molecule of claim 4, wherein the first Fab fused at its N-terminus of the Fab heavy chain to the C-terminus of the variable region of heavy chain (V_{H}) of the scFv, or the first Fab fused at its N-terminus of the Fab heavy chain to the C-terminus of the variable region of light chain (V_{L}) of the scFv.

9.-10. (canceled)

11. The dual targeting antigen binding molecule of claim 1, wherein the first and the second antigen binding moiety are fused to each other via a peptide linker, wherein the peptide linker is (GxSy)n, and the x and y are individually any integer selected from 1-5, and n is any integer selected from 1-5.

12.-13. (canceled)

14. The dual targeting antigen binding molecule of claim 1, wherein the Fc domain is a human IgG Fc domain.

15. The dual targeting antigen binding molecule of claim 14, wherein the Fc domain is a human IgG1 or IgG4 Fc domain.

16. The dual targeting antigen binding molecule of claim 15, wherein the Fc domain comprises one or more modifications promoting the association of the first and the second subunit of the Fc domain.

17. The dual targeting antigen binding molecule of claim 16, wherein in the CH3 domain of the first subunit of the Fc domain an amino acid residue is replaced with an amino acid residue having a larger side chain volume, thereby generating a protuberance within the CH3 domain of the first subunit, and in the CH3 domain of the second subunit of the Fc domain an amino acid residues is replaced with an amino acid residue having a smaller side chain volume, thereby generating a cavity within the CH3 domain of the second subunit, wherein the protuberance is protrudable into the cavity.

18. The dual targeting antigen binding molecule of claim 17, wherein in the CH3 domain of the first subunit of the Fc domain, the T366 residue is replaced with an amino acid residue having a larger side chain volume.

19. The dual targeting antigen binding molecule of claim 17, wherein in the CH3 domain of the second subunit of the Fc domain, one or more residues selected from T366, L368, and Y407 are replaced with one or more amino acid residues having a smaller side chain volume.

20. (canceled)

21. The dual targeting antigen binding molecule of claim 1, wherein the Fc domain exhibits reduced binding affinity to an Fc receptor and/or reduced effector function, as compared to a native IgG1 or IgG4 Fc domain, and wherein the Fc domain comprises one or more amino acid substitutions that reduce binding to an Fc receptor and/or effector function.

22. (canceled)

23. The dual targeting antigen binding molecule of claim 21, wherein said one or more amino acid substitutions are at one or more positions selected from the group of L/F234, L235, D265, N297 and P329.

24. The dual targeting antigen binding molecule of claim 23, wherein each subunit of the Fc domain comprises two amino acid substitutions that reduce binding to an activating Fc receptor and/or effector function wherein said amino acid substitutions are L/F234A and L235A.

25.-26. (canceled)

27. A dual targeting antigen binding molecule of claim 21, comprising an amino acid substitution at the position of S228 of IgG4.

28. (canceled)

29. A dual targeting antigen binding molecule, comprising a) an Fc domain of human IgG, consisting of a first and a second subunit capable of stable association, b) a first antigen binding moiety capable of specific binding to a T cell-activating antigen, comprising a scFv, and c) a second antigen binding moiety capable of specific binding to a target cell antigen, comprising a first Fab and a second Fab, wherein
1) the scFv, at the N-terminus of the variable region of heavy chain (V_{H}) of the scFv or at the N-terminus of the variable region of light chain (V_{L}) of the scFv, is fused to the C-terminus of the Fab heavy chain of the first Fab, and at the C-terminus of the variable region of heavy chain (V_{H}) or the variable region of light chain (V_{L}) of the scFv, is fused to the first subunit of the Fc domain comprising a substitution of T366 with an amino acid residue having a larger side chain, and
2) the second Fab, at the C-terminus of the Fab heavy chain, is fused to the second subunit of the Fc domain comprising one or more substitutions of T366, L368, and/or Y407 with an amino acid residue having a smaller side chain volume.

30.-49. (canceled)

50. The dual targeting antigen binding molecule of claim 1, wherein the T cell-activating antigen is any one selected from the group consisting of CD3, 4-1BB, PD-1 and CD40L/CD154.

51. (canceled)

52. The dual targeting antigen binding molecule of claim 1, wherein the target cell antigen is any one selected from the group consisting of: CD19, CD20, CD33, CD38, Melanoma-associated Chondroitin Sulfate Proteoglycan (MCSP), cell surface associated mucin 1 (MUC1), Epidermal Growth Factor Receptor (EGFR), HER2, Carcinoembryonic Antigen (CEA), B7-H1, B7-H3, B7-H4, Glypican-3, Mesothelin, Trophoblast glycoprotein (5T4), Transferrin receptor 1 (TfR1) and Fibroblast Activation Protein (FAP).

53.-68. (canceled)

69. The dual targeting antigen binding molecule of claim 1, wherein the T cell-activating antigen is CD3, and the target cell antigen is CD20.


SEQUENCE LISTING (28 sequences)

SEQ ID NO: 1 - DNA, 366 bp, Artificial Sequence
  The nucleotide sequence coding for VH of anti-CD20 Fab
gaagtgcagctggtggagtctgggggaggcttggtacagcctggcaggtccctgagactctcctgtgcagcctctggattcacctttaatgattatgccatgcactgggtccggcaagctccagggaagggcctggagtgggtctcaactattagttggaatagtggttccataggctatgcggactctgtgaagggccgattcaccatctccagagacaacgccaagaagtccctgtatctgcaaatgaacagtctgagagctgaggacacggccttgtattactgtgcaaaagatatacagtacggcaactactactacggtatggacgtctggggccaagggaccacggtcaccgtctcctca

SEQ ID NO: 2 - protein, 122 aa, Artificial Sequence
  The amino acid sequence of VH of anti-CD20 Fab
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 3 - protein, 8 aa, Artificial Sequence
  The amino acid sequence of CDR1 in VH of anti-CD20 Fab
GFTFNDYA

SEQ ID NO: 4 - protein, 8 aa, Artificial Sequence
  The amino acid sequence of CDR2 in VH of anti-CD20 Fab
ISWNSGSI

SEQ ID NO: 5 - protein, 15 aa, Artificial Sequence
  The amino acid sequence of CDR3 in VH of anti-CD20 Fab
AKDIQYGNYYYGMDV

SEQ ID NO: 6 - DNA, 321 bp, Artificial Sequence
  The nucleotide sequence coding for VL of anti-CD20 Fab
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgatcaccttcggccaagggacacgactggagattaaa

SEQ ID NO: 7 - protein, 107 aa, Artificial Sequence
  The amino acid sequence of VL of anti-CD20 Fab
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 8 - protein, 6 aa, Artificial Sequence
  The amino acid sequence of CDR1 in VL of anti-CD20 Fab
QSVSSY

SEQ ID NO: 9 - protein, 3 aa, Artificial Sequence
  The amino acid sequence of CDR2 in VL of anti-CD20 Fab
DAS

SEQ ID NO: 10 - protein, 9 aa, Artificial Sequence
  The amino acid sequence of CDR3 in VL of anti-CD20 Fab
QQRSNWPIT

SEQ ID NO: 11 - DNA, 357 bp, Artificial Sequence
  The nucleotide sequence coding for VH of anti-CD3 scFV (#1)
caggtgcagctggtgcagagcggcggcggcgtggtgcagcccggccgcagcctgcgcctgagctgcaaggccagcggctacaccttcacccgctacaccatgcactgggtgcgccaggcccccggcaagggcctggagtggatcggctacatcaaccccagccgcggctacaccaactacaaccagaaggtgaaggaccgcttcaccatcagcaccgacaagagcaagagcaccgccttcctgcagatggacagcctgcgccccgaggacaccgccgtgtactactgcgcccgctactacgacgaccactactcgctggactactggggccagggcacccccgtgaccgtgtcctca

SEQ ID NO: 12 - protein, 119 aa, Artificial Sequence
  The amino acid sequence of VH of anti-CD3 scFV (#1)
QVQLVQSGGGVVQPGRSLRLSCKASGYTFTRYTMHWVRQAPGKGLEWIGYINPSRGYTNYNQKVKDRFTISTDKSKSTAFLQMDSLRPEDTAVYYCARYYDDHYSLDYWGQGTPVTVSS

SEQ ID NO: 13 - protein, 8 aa, Artificial Sequence
  The amino acid sequence of CDR1 in VH of anti-CD3 scFV
GYTFTRYT

SEQ ID NO: 14 - protein, 8 aa, Artificial Sequence
  The amino acid sequence of CDR2 in VH of anti-CD3 scFV
INPSRGYT

SEQ ID NO: 15 - protein, 12 aa, Artificial Sequence
  The amino acid sequence of CDR3 in VH of anti-CD3 scFV
ARYYDDHYSLDY

SEQ ID NO: 16 - DNA, 318 bp, Artificial Sequence
  The nucleotide sequence coding for VL of anti-CD3 scFV
gacatccagatgacccagagccccagcagcctgagcgccagcgtgggcgaccgcgtgaccatcacctgcagcgccagcagcagcgtgagctacatgaactggtaccagcagacccccggcaaggcccccaagcgctggatctacgacaccagcaagctggccagcggcgtgcccagccgcttcagcggcagcggcagcggcaccgactacaccttcaccatcagcagcctgcagcccgaggacatcgccacctactactgccagcagtggagcagcaaccccttcaccttcggccagggcaccaagctgcagatcacc

SEQ ID NO: 17 - protein, 106 aa, Artificial Sequence [sequence omitted]

SEQ ID NO: 18 - protein, 5 aa, Artificial Sequence
  The amino acid sequence of CDR1 in VL of anti-CD3 scFV
SSVSY

SEQ ID NO: 19 - protein, 3 aa, Artificial Sequence
  The amino acid sequence of CDR2 in VL of anti-CD3 scFV
DTS

SEQ ID NO: 20 - protein, 9 aa, Artificial Sequence
  The amino acid sequence of CDR3 in VL of anti-CD3 scFV
QQWSSNPFT

SEQ ID NO: 21 - DNA, 357 bp, Artificial Sequence
  The nucleotide sequence coding for VH of anti-CD3 scFV (#2)
caggtgcagctggtgcagagcggcggcggcgtggtgcagcccggccgcagcctgcgcctgagctgcaaggccagcggctacaccttcacccgctacaccatgcactgggtgcgccaggcccccggcaagggcctggagtggatcggctacatcaaccccagccgcggctacaccaactacaaccagaaggtgaaggaccgcttcaccatcagccgcgacaatagcaagaacaccgccttcctgcagatggacagcctgcgccccgaggacaccggcgtgtacttctgcgcccgctactacgacgaccactactcgctggactactggggccagggcacccccgtgaccgtgtcctca

SEQ ID NO: 22 - protein, 119 aa, Artificial Sequence
  The amino acid sequence of VH of anti-CD3 scFV (#2)
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSS

SEQ ID NO: 23 - DNA, 29 bp, Artificial Sequence
  Forward primer
ggtcagcctgacctgcctggtcaaaggct

SEQ ID NO: 24 - DNA, 28 bp, Artificial Sequence
  Reverse primer
tggttcttggtcatctcctcctgggatg

SEQ ID NO: 25 - DNA, 26 bp, Artificial Sequence
  Forward primer
cttcttcctctacagcaggctaaccg

SEQ ID NO: 26 - DNA, 27 bp, Artificial Sequence
  Reverse primer
gagccgtcggagtccagcacgggaggc

SEQ ID NO: 27 - DNA, 40 bp, Artificial Sequence
  Forward primer
aaccaggtcagcctgtggtgcctggtcaaaggcttctacc

SEQ ID NO: 28 - DNA, 38 bp, Artificial Sequence
  Reverse primer
cttggtcatctcctcctgggatgggggcagggtgtaca